SEQUENCE LISTING (25 sequences)

SEQ ID NO: 1 - DNA, 4968 bp, Equus caballus
  exon (1)..(4968)
atgcccattgtcaaagcaaacctgaaaagctcccactgtggcgtttttggagtgactgtggataccagtcatccagcttcctgccaggctgaggaggaagaggctggcggaggcctggctgcagcctccccgctccactgctgcggcaggtgttgctgtggccagctcaccaaccagcatattccccctctgccaagtgtgacatcgagcaaaaatggagaagaaaataaacaggtggagactcagcctgaaaaatggtctgttgccaaacacacccagagctacccaacagattcctatgggattcttgagttccagggtggcggatactccaataaagccatgtatatccgcgtatcctatgacaccaagccagattcactgcttcatctcatggtgaaagattggcatctggaactcccgaagcttttaatatctgtgcacggaggcctccaaaactttgagatgcagcctaagctgaaacaggtctttgggaaaggtctgatcaaggccgccatgaccaccggggcctggatcttcactgggggcgtcagcaccggtgtcatcagccatgtaggggatgccttgaaagaccactcttccaagtccagaggccgagtttgtgctataggaattgctccgtggggcatcgtggagaataaggaagacctggttggaaaggatgtaacgagagtgtaccagaccatgtctaaccctctaagtaagctctctgtgctcaacaactcccacactcacttcatcctggctgacaacggcaccctgggcaaatatggcgccgaggtgaagctgagaaggcagctagaaaagcacatctctctgcaaaagatcaacacaagactggggcagggtgtgcccctcgtgggcctcgtggtggaagggggccctaacgtggtttccatcgtcttggagtatctccgagaagacccaccagtccccgtggtggtctgcgacggcagcggacgagcctcggacatcctgtcttttgcacacaagtactgtgaagaaggagggataataaacgaatctctcagggatcagcttctagttaccattcagaaaacatttaattacaacaaggcacaatcgcatcagctgtttgcaattataatggagtgcatgaaaaagaaagaactcgtcaccgtgtttagaatgggtgccgagggtcagcaagacatcgagatggcaattttaactgccctgctgaaaggaacaaacgcatcggcgccagaccaactgagcttggcgctggcttggaaccgcgttgacatagcacgaagccagatcctcgtctttggggcccactggccgcccctgggaagcctggcccccccgacggatagcaaggccacagagaaggagaagaagccgcccacagcgaccaccaaggcaggaagaggaaaaggaaaagggaagaagaaagggaaagtgaaagaggaggtggaggaagaaactgatccccggaagatcgagctgctgaactgggtgaatgctctggagcaagccatgctggacgctttggtcttagatcgagtcgactttgtgaagctcctgattgaaaatggagtgaacatgcaacactttcttaccattccaaggctggaagagctttataacacaaggctgggtccaccaaacacacttcatttgctggtgagagacgtgaaaaagggcaaccttcctcccgactaccacatcagcctcatagacatagggctcgtgctggagtacctcatgggaggcgcctaccgctgcaactatactcggaagagctttcggaccctttacaacaacttgtttggacccaagaggcctaaagcccttaaacttctggggatggaagatgatgagcctccagcaaaagggaagaaaaagaaaaagaagaaaaaggaggaagagatcgacatcgatgtggacgatcctgctgtgagccgtttccagtaccccttccacgagctgatggtgtgggctgttctgatgaaacgccaaaaaatggccgtcttcctgtggcagcgaggcgaggagagcatggccaaggccctggtggcctgtaagctctacaagtccatggcccatgagtcctccgagagcgagctggtggacgacatctcccaggatctggacaacaattcaaaagactttggccagctggctgtggagctgttggaccaatcctataagcacgacgagcagatcgccatgaaactgctcacctatgagctaaaaaactggagcaactcgacctgcctcaagctggctgttgcagccaaacaccgggacttcattgctcacacctgcagccagatgctgctgaccgacatgtggatgggacggctgcgcatgaggaaaaatcctggcctaaaggttataatggggattcttcttccccccaccatcttattcttggaatttcgcacgtatgatgacttctcatatcaaacatccaaggaaaatgaagatggcaaagaaaaagaagaggagactatggatgcgaatgcagatgctggctccagaaagggggatgaagagaatgagcacaaaaagcaaagaagtattcccattggcacaaagatctgtgaattctataatgctcccattgtcaagttctggttttacacaatatcgtacttaggttacttactgctcttcaactacgtcatcctggtgcggatggaccgctggccctgtctccaggagtggatcgtcatctcctacatcgtgagcctggccttagagaaaatacgagagatcctaatgtccgaaccgggcaaactcagccagaaaataaaggtttggcttcaggagtactggaacatcacggatctcgtggccatttccatgttcatgattggagcaatccttcgcctacagaagcagccgtacatgggctacggccgagtcatctactgtgtggatatcatcttctggtacatccgcgtcctggacatctttggtgttaacaagtatctggggccctacgtgatgatgattggaaagatgatgattgacatgctgtattttgtggtcatcatgctggttgtgctgatgagttttggagtagcccgtcaagccattttgcacccagatgaggagccgtcttggaaactggcccgaaacatcttctacatgccctactggatgatctacggagaggtgtttgcagaccagatagacctctacgctatggaaattaatcctccttgtggtgacaaccagtacgatgaggacggcaagcggctccctccgtgtatcccaggtgcctggctcactccggccatcatggcgtgctacctcctggtggccaacatcctgctggtgaacctgctcattgcagtttttaacaataccttctttgaagtaaagtcaatatccaaccaagtgtggaaattccagcgctatcaactgattatgacattccatgaccggccagtcctccctccaccaatgatcattttaagccacatctacataatcattatgcgtcttagtggtcgctgtaggaagaaaagagaaggggaccaagacgaacaggatcgtggactgaagctgtttctcagcgaggaggagctcaagagactgcatgagttcgaggagcagtgcgtgcaggagcacttccaagagaaggaggacgagcagcagtcgtccagtgacgagcgcatccgcgtcacttgtgagagggttgaaaatatgtcaatgaggttggaagaaatcaatgaacgagaaaattttatgaaaacttccctacagactgttgaccttcgactttcccaactagaagaattatctagcagaatggtgaatgctctggaaaatcttgcaggaattgacaggtccgacctgatacagacacggtcccgagcttcttctgtatgcgatgcaacttttcttctaaggcagagcagcctcagcagcgccgacggctacagcatgtgtagatatcattttaatggagaggagctgttgtgtgaggatacttctgtctccaagccaccagggacaggattcaggaaaaaagccggttccttccgtatgaaggaagagaaggacccaaaaacacatctggtaccagagcgtcagggcagccttcaccttctatccagcacaagcacgccagcaacgccagagtgcagtcgacttgcattcgacaacgtaaagtacacctcagagccgaaattaggtccagatattgggatttcagctgaagatgatgaaaggcgggcagactccagaagagaagaaactatttccccaagtttaaatcaaacagatgttaagaacggagagaacaaatcagattttcaaaacactcagctaaccgtggaaaccacagagatagaaggcactatttcctatcctctggaagaaacaaaaatgacacgctattaccccgaggaaacgctcaatgcgtgtaaaacaatgaagtccagaagctttgtatattcccgtggaagaaagctggtcagaggggctaacaactggagtgcagagtacagtacagtcacggaccaagcaggcccctctacaattcagcaatggactacagaatggaaataccaagttagaaagatcacacgttctcacagcacggatattcctttgattgtgtcagaagctgcaatgcaagctgagcggaaagaacaatttacagataccgaagatgaaaactgtgtttctgaagcagtccctcaaacttctcatttatctctagctgttactgacagaactggaaaggaaaacttactgtctgtgaaaacagaccaaactttgggattcccatctttaaggtcaaaaagcttacacggccatcctagaaatgctaaaggctttcagggaaaattagatagatctgggcatgccagcagtgcaagtaacttagtagttgtgtctggaattacagtggaagaacagaaggccaagcaagagaaaacttccacagaaactgaatgctaa

SEQ ID NO: 2 - DNA, 17 bp, Equus caballus
gccgtcttcccctccat

SEQ ID NO: 3 - DNA, 21 bp, Equus caballus
gcccacgtatgagtccttctg

SEQ ID NO: 4 - DNA, 23 bp, Equus caballus
ggcaccagggcgtgatggtgggc

SEQ ID NO: 5 - DNA, 20 bp, Equus caballus
gacgacatctcccaggatct

SEQ ID NO: 6 - DNA, 20 bp, Equus caballus
tgctcgtcgtgcttatagga

SEQ ID NO: 7 - DNA, 25 bp, Equus caballus
attcaaaagactttggccagctggc

SEQ ID NO: 8 - DNA, 23 bp, Equus caballus
agatcaaggaaagttctggcagt

SEQ ID NO: 9 - DNA, 18 bp, Equus caballus
ctggagcagcgtggaatc

SEQ ID NO: 10 - DNA, 28 bp, Equus caballus
aagctactctgtgaacctcagcagccat

SEQ ID NO: 11 - DNA, 22 bp, Equus caballus
atatgggaacaacacacagtga

SEQ ID NO: 12 - DNA, 20 bp, Equus caballus
ggtcctcctttcagcacatc

SEQ ID NO: 13 - DNA, 25 bp, Equus caballus
cttcacagggctcctggatttggat

SEQ ID NO: 14 - DNA, 21 bp, Equus caballus
tgtcagatttcgctttgatga

SEQ ID NO: 15 - DNA, 20 bp, Equus caballus
ggtctgttggctgggaataa

SEQ ID NO: 16 - DNA, 25 bp, Equus caballus
tcaggtcctgaaagtgccaaaaagg

SEQ ID NO: 17 - DNA, 20 bp, Equus caballus
cagactttgttcggtccaca

SEQ ID NO: 18 - DNA, 20 bp, Equus caballus
agtcactcagagcggctgtc

SEQ ID NO: 19 - DNA, 25 bp, Equus caballus
agaacctggtctggccagagacctg

SEQ ID NO: 20 - DNA, 20 bp, Equus caballus
tccaaagttcccttccatca

SEQ ID NO: 21 - DNA, 20 bp, Equus caballus
tgccagaatgttgaccatgt

SEQ ID NO: 22 - DNA, 20 bp, Equus caballus
gactgagcgtatgtgcgtgt

SEQ ID NO: 23 - DNA, 20 bp, Equus caballus
ctcctgtctgagtggcttca

SEQ ID NO: 24 - DNA, 20 bp, Equus caballus
taaccatgaccagtcctatc

SEQ ID NO: 25 - DNA, 20 bp, Equus caballus
gcaccagtctatcatgtgtg

(19) United States
(12) Patent Application Publication
(10) Pub. No.: US 2011/0104687 A1
(43) Pub. Date: May 5, 2011

(54) Methods and Kits for Detecting Congenital Stationary Night Blindness and Selecting Different Coat Patterns

(76) Inventors: Bruce Grahn, Saskatchewan (CA); Lynne Sandmeyer, Saskatchewan (CA); George Forsyth, Saskatchewan (CA); Rebecca Bellone, Plant City, FL (US)

(21) Appl. No.: 12/919,598

(22) PCT Filed: Feb. 27, 2009
(86) PCT No.: PCT/CA2009/000240

§ 371 Date: Nov. 30, 2010

Related U.S. Application Data
(60) Provisional application No. 61/031,807, filed on Feb. 27, 2008.

(51) Int. Cl. C12Q 1/68; C07H 21/04; C07K 16/18

(52) U.S. Cl. 435/6; 536/24.31; 530/387.1; 536/24.33

(57) ABSTRACT

The present application describes biomarkers and methods useful for screening for, diagnosing or detecting congenital stationary night blindness in a subject. The present application also provides methods for selecting or detecting horse coat patterns.

This is a national stage application of PCT/CA2009/000240 filed on Feb. 27, 2009 which claims priority from U.S. provisional application 61/031,807 filed on Feb. 27, 2008, both of which are incorporated herein by reference in their entirety.

### FIELD

The application relates to methods and kits for screening, detecting or diagnosing congenital stationary night blindness. Further, the application relates to methods and kits for screening or detecting horse coat patterns.

### BACKGROUND

Coat color has been a fascinating topic of genetic discussion and discovery for over a century. The pigment genes of mice were one of the first genetic systems to be explored through breeding and transgenic studies. To date at least 127 loci involved in pigmentation have been described (Silvers, 1979; Bennett and Lamoreux, 2003). Often the genes that affect pigmentation in the skin and hair influence other body systems, and many such genes have been studied in several different mammals. One of the many extensively studied examples is oculocutaneous albinism type 1; a developmental disorder in humans that affects pigmentation in the skin and as well as eye development. This disease is caused by mutations in the tyrosinase gene (TYR), which is involved in the first step of melanin production (Toyofuko et al. 2001; Ray et al. 2007).

Horses (Equus Caballus) are among the domesticated animals valued by breeders and enthusiasts for their variety and beauty of coat color and patterns. The genetic mechanisms involved in several different variations of coloration and patterning in horses have been reported including; chestnut, frame overo, cream, black, silver dapple, sabino-1 spotting, tobiano spotting and dominant white spotting (Marklund et al. 1996; Metallinos et al. 1998; Mariat et al. 2003; Reider et al. 2003; Brunberg et al. 2006; Brooks and Bailey 2005; Brooks et al., 2007; Haase et al. 2007). The mechanism behind appaloosa spotting, a popular coat pattern occurring in several breeds of horses, remains to be elucidated. Likewise, although there are several inherited ocular diseases reported in the horse (cataracts, glaucoma, anterior segment dysgenesis, and congenital stationary night blindness), the modes of inheritance, genetic mutations, and the pathogenesis of these ocular disorders remain unknown.

Appaloosa spotting is characterized by patches of white in the coat that tend to be symmetrical and centered over the hips. In addition to the patterning in the coat, appaloosa spotted horses have three additional pigmentation traits; striped hooves, readily visible nonpigmented sclera around the eye, and mottled pigmentation around the anus, genitalia, and muzzle (Sponenberg and Beaver 1983). The extent of spotting varies widely among individuals, resulting in a collection of patterns which are termed the “leopard complex” (Sponenberg et al. 1990). This variation encompasses a broad spectrum of patterns; including those possessing very minimal patches on the rump (known as a “lace blanket”), a white body with many oval or round pigmented spots dispersed throughout (known as “leopard”, from which the genetic locus is named), as well as a nearly complete depigmentation (known as “fewspot”) (FIG. 1). A single autosomal dominant gene, Leopard Complex (LP), is thought to be responsible for the inheritance of these patterns and associated traits, while modifier genes are thought to play a role in determining the amount of white patterning that is inherited (Miller 1965; Sponenberg et al. 1990; Archer and Bellone unpublished data). Horses that are homozygous for appaloosa spotting (LP/LP) tend to have fewer spots on the white patterned areas; these horses are known as “fewspots” (largely white body with little to no spots) and “snowcaps” (white over the croup and hips with little to no spots) (Sponenberg et al. 1990; Lapp & Carr 1998) (FIG. 1).

A whole genome scanning panel of microsatellite markers was used to map LP to a 6 cM region on ECA1 (Terry et al. 2004). Prior to the sequencing of the equine genome, two candidate genes Transient Receptor Potential Cation Channel, Subfamily M, Member 1 (TRPM1) and Oculoctaneous Albinism Type II (OCA2) were suggested based on comparative phenotypes in humans and mice (Terry et al. 2004). Both TRPM1 and OCA2 were FISH mapped to ECA1, to the same interval as LP (Bellone et al. 2006a). One SNP in the equine OCA2 gene has been ruled out as the cause for appaloosa spotting (Bellone et al. 2006b).

TRPM1, also known as Melastatin 1 (MLSN1), is a member of the transient receptor potential (TRP) channel family. Channels in the TRP family may permit Ca21 entry into hyperpolarized cells, producing intracellular responses linked to the phosphatidylinositol and protein kinase C signal transduction pathways (Clapham et al. 2001). TRPs are important in cellular and somatosensory perception (Nilius, 2007). Defects in a light-gaited TRP channel results in a loss of phototransduction in Drosophila (reviewed in Kim, 2004). Although the specific function of TRPM1 has yet to be described, cellular sensation and intercellular signaling is vital for normal melanocyte migration (reviewed in Steingrimsson et al. 2006). In mice and humans, the promoter region of this gene contains four consensus binding sites for a melanocyte transcription factor, MITF (Hunter et al. 1998; Zhiqi et al. 2004). One of these sites, termed an M-box, is unique to melanocytic expression (Hunter et al. 1998). TRPM1 is downregulated in highly metastatic melanoma cells, suggesting that this protein plays an important role in normal melanogenesis (Duncan et al. 1998).

Mutations in the OCA2 gene (also P, or pink-eyed dilution) cause hypopigmentation phenotypes in mice (Gardner et al. 1992). Similarly, in humans, mutations in OCA2 cause the most common form of albinism (Lee et al. 1994). Additionally, other mutations in this gene are thought to be responsible for the variation in human eye color (Duffy et al. 2007; Eiberg et al. 2008). It is believed that during melanogenesis this protein functions to control intramelanasomal pH and aids in tryosinase processing (Sturm et al. 2001; Ni-Komatsu and Orlow 2005).

An association of homozygosity for LP and congenital stationary night blindness (CSNB) was recently documented (Sandmeyer et al. 2007). CSNB is characterized by a congenital and non-progressive scotopic visual deficit (Witzel et al. 1977, 1978; Rebhun et al. 1984). Affected horses may exhibit apprehension in dimly lit conditions and may be difficult to train and handle in phototopic (light) and scotopic (dark) conditions (Witzel et al. 1977, 1978; Rebhun et al. 1984). Affected animals occasionally manifest a bilateral dorsomedial strabismus (improper eye alignment) and nystagmus (involuntary eye movement) (Rebhun et al. 1984; Sandmeyer et al. 2007). CSNB is diagnosed by an absent b-wave and a depolarizing a-wave in scotopic (dark-adapted) electroretinography (ERG) (FIG. 2). This ERG pattern is known as a “negative ERG” (Witzel et al. 1977). No morphological or ultrastructural abnormalities have been detected in the retinas of horses with CSNB (Witzel et al. 1977; Sandmeyer et al. 2007). A similar “negative ERG” is seen in the Schubert-Bornshein type of human CSNB (Schubert and Bornshein 1952; Witzel et al. 1978). This type of CSNB is thought to be caused by a defective neural transmission within the retinal rod pathway (Witzel et al. 1977, 1978; Sandmeyer et al. 2007). Neural transmission is complex and the mechanism of the transmission defect in CSNB is not reported. Rod photoreceptors are most sensitive under scotopic conditions. In the dark, these cells exist in a depolarized state. They hyperpolarize in response to light, and signaling occurs through reductions in glutamate release (Stryer 1991). This hyperpolarization is responsible for the a-wave of the electroretinogram. Normally this results in stimulation of a population of bipolar cells, the ON bipolar cells. The glutamate receptor of the ON bipolar cells is a metabotropic glutamate receptor (MGIuR6) and this receptor is expressed only in the retinal bipolar cell layer (Nomura et al. 1994; Nakanishi et al. 1998). The MGIuR6 receptors sense the reduction in synaptic glutamate and produce a response that depolarizes the ON bipolar cell (Nakanishi et al. 1998). This depolarization is responsible for the b-wave of the electroretinogram. The ERG characteristics of the Schubert-Bornshein type of CSNB are consistent with a failure in depolarization of the ON bipolar cell (Sandmeyer et al. 2007).

### SUMMARY OF THE DISCLOSURE

The inventors of the present application have identified a biomarker that is differentially expressed in subjects having or not having congenital stationary night blindness (CSNB). Specifically, the biomarker is the TRPM1 gene. Further, the inventors have identified that the biomarker is associated with coat phenotype in horses and can be used to select or detect coat patterns.

Accordingly, one aspect of the application is a method of screening for, detecting or diagnosing congenital stationary night blindness in a subject by determining the level of a biomarker product in a sample from the subject. Another aspect of the application is a method of detecting or selecting coat patterns in a horse by determining the level of a biomarker product in a sample from the horse.

The inventors have also identified single nucleotide polymorphisms (SNP) associated with Leopard complex (LP/LP or LP) and CSNB. Accordingly, another aspect of the application is a method of screening for, detecting or diagnosing congenital stationary night blindness (CSNB) in a subject by determining the presence of at least one SNP allele associated with CSNB in the TRPM1 gene.

In yet another aspect, the application provides a method of detecting or selecting coat patterns in a horse by determining the presence of at least one SNP allele associated with different coat patterns or leopard complex (LP) in the TRPM1 gene.

The application also provides compositions and kits that can be used for screening for, detecting or diagnosing congenital stationary night blindness. Further, the application provides compositions and kits that can be used to select or detect coat patterns.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a series of photographs of horses displaying different appaloosa coat color patterns: (a) lace blanket (LP/lp); (b) spotted blanket (LP/lp); (c) leopard (LP/lp); (d) snowcap blanket (LP/LP); and (e) fewspot (LP/LP).

FIG. 2 is a scotopic electroretinogram from an lp/lp Appaloosa (left) and an LP/LP Appaloosa with CSNB (right). Note the absence of a b-wave in the ERG tracing from the LP/LP horse. (50 msec, 100 mV).

FIG. 3 is a schematic of the genomic map highlighting the genes tested for differential expression within the LP candidate region on ECA1.

FIG. 4 shows retinal and skin gene expression for five genes in the LP candidate region normalized to β-actin. Relative mRNA expression is represented as a log 2 relative expression ratio (means±SE). (A) CSNB affected (LP/LP) and CSNB unaffected (LP/lp) retinal RNA samples. Data are expressed as relative to CSNB unaffected (lp/lp) mRNA levels. (B) Pigmented and unpigmented skin samples of homozygous (LP/LP) and heterozygous (LP/lp) horses. Data are expressed as relative to non-appaloosa (lp/lp) mRNA levels. An asterisk indicates significant results (P<0.05).

FIG. 5 shows fine mapping the Leopard Complex Gene (LP) and CSNB. SNP association with LP and CSNB is represented as −logP. The region with the strongest association is highlighted in light. The deeper highlight reflects the SNP with the highest association. Corresponding annotated genes are shown below the SNP data.

FIG. 6 shows the alignment of the chromatograms from one LP/LP and one lp/lp horse illustrating SNP detected in intron 11-12 108249239 G>A.

FIG. 7 shows a 1.5% agarose gel visualized with ethidium bromide, illuminated by ultraviolet light, displaying the BsmF1 PCR-RFLP products. This RFLP was designed with an internal cut site that generated a 529-bp and 100-bp product. This enzyme also recognized the 108249293 G allele generating a product that was 362-bp and one that was 167-bp. Lane 1 contained the size standard, Lane 2-9 contained DNA from 8 different Appaloosas and Lane 10-12 contained DNA from the 3 positive controls.

### DETAILED DESCRIPTION OF THE INVENTION

### (A) Biomarker

The present application discloses a biomarker, which is differentially expressed in subjects having or not having congenital stationary night blindness. Further, the application discloses that the biomarker is differentially expressed in horses with different coat patterns.

The term “biomarker” as used herein refers to any type of molecule that can be used to distinguish subjects with or without congenital stationary night blindness or that can be used to distinguish horses with different coat patterns. In a specific embodiment, the biomarker is TRPM1 and includes, without limitation, all known TRPM1 molecules, including naturally occurring variants, and including those deposited in Genbank with accession number XM_{—}001492235.1 (SEQ ID NO:1) and accession number NM 002420.

The term “differentially expressed” or “differential expression” as used herein refers to a difference in the level of expression of the biomarker disclosed herein (i.e. TRPM1) that can be assayed by measuring the level of expression of the biomarker products.

The term “biomarker products” as used herein refers to RNA and/or protein expressed by the biomarker described in the present application. In a specific embodiment, the biomarker is the TRPM1 gene and the biomarker product is a TRPM1 gene product. In the case of RNA, it refers to RNA transcripts transcribed from the TRPM1 gene. The term “RNA product” as used herein includes mRNA transcripts, and/or specific spliced variants of mRNA. In the case of protein, it refers to proteins translated from the RNA transcripts transcribed from the TRPM1 gene. The term “protein product” includes proteins translated from the RNA products of the TRPM1 gene. Protein products include expressed, secreted, cleaved, released, and shed protein products. The term biomarker products also includes full length or fragments of TRPM1 RNA or proteins.

### (B) Single Nucleotide Polymorphisms

The present inventors have identified single nucleotide polymorphisms associated with horse coat patterns (LP genotype) in the TRPM1 gene. The phrase “single nucleotide polymorphism or SNP associated with LP” as used herein refers to single nucleotide polymorphisms in the TRPM1 gene that are associated with LP genotype. In one embodiment, the single nucleotide polymorphism is located at position 108249293 of ECA1 (horse chromosome 1) and the allele associated with LP is A. In another embodiment, the single nucleotide polymorphism is located at position 108267503 of ECA1 and the allele associated with LP is C. In yet another embodiment, the single nucleotide polymorphism is located at position 108246967 of ECA1, and the allele associated with LP is T. In a further embodiment, the single nucleotide polymorphism is located at position 108247024 of ECA1, and the allele associated with LP is T. In another embodiment, the single nucleotide polymorphism is selected from the SNPs listed in Table 8. In one embodiment, the single nucleotide polymorphism is located at position 108370091 of ECA1 and the allele associated with LP is a T nucleotide.

The present inventors have also identified single nucleotide polymorphisms associated with CSNB in the TRPM1 gene. The phrase “single nucleotide polymorphism or SNP associated with CSNB” as used herein refers to single nucleotide polymorphisms in the TRPM1 gene that are associated with CSNB. In one embodiment, the single nucleotide polymorphism is located at position 108249293 of ECA1 (horse chromosome 1) and the allele associated with CSNB is A. In another embodiment, the single nucleotide polymorphism is located at position 108267503 of ECA1 and the allele associated with CSNB is C. In yet another embodiment, the single nucleotide polymorphism is located at position 108246967 of ECA1, and the allele associated with CSNB is T. In a further embodiment, the single nucleotide polymorphism is located at position 108247024 of ECA1, and the allele associated with CSNB is T. In another embodiment, the at least one SNP associated with CSNB is selected from the SNPs listed in Table 9. In one embodiment, the single nucleotide polymorphism is located at position 108370091 of ECA1 and the allele associated with CSNB is a T nucleotide. In another embodiment, the single nucleotide polymorphism is located at position 108370150 of ECA1 and the allele associated with CSNB is a C nucleotide.

The term “single nucleotide polymorphism” or SNP as used herein refers to a genetic variation in the DNA sequence that occurs at a single nucleotide position on ECA1.

The term “ECA1” as used herein refers to horse chromosome 1.

### (B) Methods

### (i) Definitions

The term “subject” as used herein refers to any member of the animal kingdom. In one embodiment, the subject is a mammal. In another embodiment, the subject is a human being. In a further embodiment, the subject is a horse.

The term “horse” as used herein includes all breeds, including, without limitation, Appaloosa, Noriker, Knabstrubber, and the British spotted pony. In one embodiment, the breed is Appaloosa. In another embodiment, the breed is Knabstrubber.

The term “difference in the level of expression” refers to an increase or decrease in the measurable expression level of a given biomarker as measured by the amount of biomarker product in a sample as compared with the measurable expression level of a given biomarker in a second sample. The term can also refer to an increase or decrease in the measurable expression level of a given biomarker in a population of samples as compared with the measurable expression level of a biomarker in a second population of samples. In one embodiment, the differential expression can be compared using the ratio of the level of expression of a given biomarker or biomarkers as compared with the expression level of the given biomarker or biomarkers of a control, wherein the ratio is not equal to 1.0. For example, an RNA or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment, the differential expression is measured using p-value. For instance, when using p-value, a biomarker is identified as being differentially expressed as between a first and second population when the p-value is less than 0.1, preferably less than 0.05, more preferably less than 0.01, even more preferably less than 0.005, the most preferably less than 0.001.

The term “level” as used herein refers to a quantity of biomarker product that is detectable or measurable in a sample.

The term “control” as used herein refers to a sample from a subject or a group of subjects who are either known as having a particular trait or not having a particular trait. The control may also be a reference standard. A person skilled in the art will appreciate that the control will depend on the diagnostic or screening assay.

(ii) Methods to Screen for, Diagnose or Detect Congenital Stationary Night Blindness

One aspect of the present application is a method of screening for, diagnosing or detecting congenital stationary night blindness in a subject comprising the steps:

(a) determining the level of a biomarker product in a sample from the subject; and

(b) comparing the level of biomarker product in the sample with a control,

wherein detecting differential expression of the biomarker product between the subject and the control is indicative of congenital stationary night blindness in the subject.

In another aspect, the application provides a method of screening for, diagnosing or detecting congenital stationary night blindness in a subject comprising determining the presence of at least one SNP associated with CSNB. In one embodiment, the SNP associated with CSNB is located at position 108249293 of ECA1 (horse chromosome 1) and the allele associated with CSNB is A. In another embodiment, the SNP associated with CSNB is located at position 108267503 of ECA1 and the allele associated with CSNB is C. In yet another embodiment, the SNP associated with CSNB is located at position 108246967 of ECA1, and the allele associated with CSNB is T. In a further embodiment, the SNP associated with CSNB is located at position 108247024 of ECA1, and the allele associated with CSNB is T. In one embodiment, the at least one SNP associated with CSNB is selected from the SNPs on ECA1 listed in Table 9. In one embodiment, the SNP associated with CSNB is located at position 108370091 of ECA1, wherein the allele associated with CSNB is a T nucleotide. In another embodiment, the SNP associated with CSNB is located at position 108370150 of ECA1, wherein the allele associated with CSNB is a C nucleotide.

The phrase “screening for, diagnosing or detecting congenital stationary night blindness” refers to a method or process of determining if an individual has or does not have congenital stationary night blindness, and includes determining the grade or severity of congenital stationary night blindness.

The term “congenital stationary night blindness” as used herein refers to a non-progressive, inherited retinal disorder that is characterized by night blindness, decreased visual acuity, myopia, nystagmus and strabismus. It is diagnosed by an absent b-wave and depolarizing a-wave on an electroretinograph (ERG). The term also includes the Schubert-Bornshein type of human congenital stationary night blindness.

In one embodiment, the control is from a subject that is known to have congenital stationary night blindness. In another embodiment, the control is from a subject known not to have congenital stationary night blindness. The control can also be a pre-determined reference standard.

In a specific embodiment, the control is from a normal, healthy subject. For example, the control is from a subject known not to have congenital stationary night blindness. If the level of biomarker product in the sample from the subject is lower than the normal control, then this is indicative that the subject has congenital stationary night blindness.

The term “sample” as used herein refers to any fluid, cell or tissue sample from a subject which can be assayed for gene expression products, particularly genes differentially expressed in individuals having or not having congenital stationary night blindness. In one embodiment, the sample is from the eye, such as the retina or retina pigment epithelium. In another embodiment, the sample is a skin sample. If the subject has a variation in skin pigmentation, then the skin sample can be pigmented or unpigmented skin. In a further embodiment, the sample is hair. In an additional embodiment, the sample is blood or serum. The application also contemplates prenatal screening. Thus, the sample can be from a fetus.

It is contemplated that the methods described herein can be used in combination with other methods of screening for, diagnosing or detecting congenital stationary night blindness. For example, the method can be used in combination with determining the genotype for LP of the subject.

(iii) Methods to Detect or Select Coat Patterns in Horses

One aspect of the present application is a method of detecting or selecting different coat patterns in a horse comprising the steps:

(a) determining the level of a biomarker product in a sample from the horse; and

(b) comparing the level of biomarker product in the sample with a control,

wherein detecting differential expression of the biomarker product in the sample compared to the control is indicative of a different coat pattern.

In another aspect, the application provides a method of detecting or selecting different coat patterns in a horse comprising determining the presence of at least one SNP associated with LP. In one embodiment, the single nucleotide polymorphism is located at position 108249293 of ECA1 (horse chromosome 1) and the allele associated with LP is A. In another embodiment, the single nucleotide polymorphism is located at position 108267503 of ECA1 C. In yet another embodiment, the single nucleotide polymorphism is located at position 108246967 of ECA1, and the allele associated with LP is T. In a further embodiment, the single nucleotide polymorphism is located at position 108247024 of ECA1, and the allele associated with LP is T. In another embodiment, the at least one SNP associated with LP is selected from the SNPs on ECA1 listed in Table 8. In one embodiment, the SNP associated with LP is located at position 108370091 of ECA1, wherein the allele associated with LP is a T nucleotide.

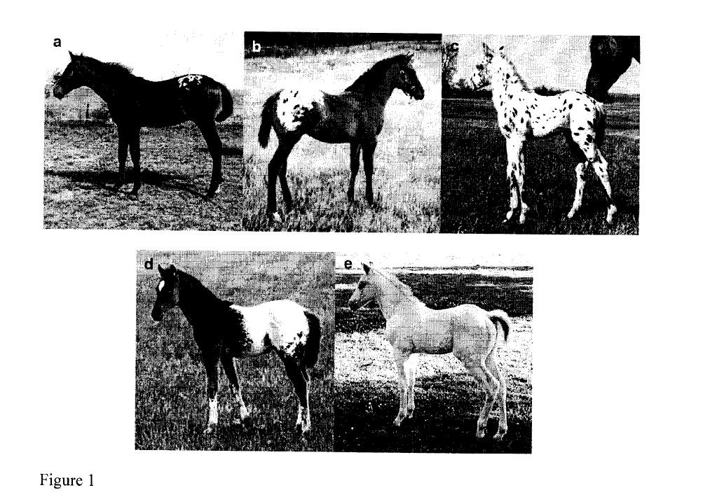

The phrase “different coat patterns” as used herein refers to variations in coat color, coat spotting, and coat patterns. Embodiments of different coat patterns are shown in FIG. 1.

The phrase “detecting or selecting different coat patterns” refers to a method or process of determining if a horse has or does not have a specific coat pattern, and includes determining the type of coat pattern.

In one embodiment, the control is from a horse that is known to have a specific coat pattern or genotype. The control can also be a pre-determined reference standard.

In one embodiment, the method disclosed herein can be used to identify the carriers of the recessive lp factor (LP/lp). As explained previously, a single autosomal dominant gene, Leopard Complex (LP), is responsible for inheritance of these coat patterns. Horses homozygous for Appaloosa spotting (LP/LP) have fewer spots on the white patterned areas than heterozygotes (LP/lp). Inheritance of the Leopard complex in Appaloosa horses can be in three forms: (1) LP/LP=few or no dark spots; (2) LP/lp=carrier (plentiful spotting); (3) lp/lp=true solid coat.

In an example embodiment, the control has the lp/lp genotype and samples are tested for the downregulation of TRPM1 as a marker for homozygous LP/LP horses.

The term “sample” as used herein refers to any fluid, cell or tissue sample from a horse which can be assayed for gene expression products, particularly genes differentially expressed in horses with different coat patterns. In one embodiment, the sample is a skin sample. If the horse has a variation in skin pigmentation, then the skin sample can be pigmented or unpigmented skin. In a further embodiment, the sample is hair. In an additional embodiment, the sample is blood or serum. The application also contemplates prenatal screening, so the sample can be from a fetus of a horse.

It is contemplated that the methods described herein can be used in combination with other methods to select or detect horse coat patterns, including genotyping and/or phenotypic observations. As one example, the methods described herein can be used in combination with genetic testing of horse coat colour offered by UC Davis Veterinary Genetics Lab; Genetic Technologies Corp., Victoria, Australia; and/or VetGen in Ann Arbor, Mich. In another embodiment, the method can be used in combination with determining the LP genotype of the horse.

### (iv) Agents to Detect Biomarker Products

The level of biomarker product is optionally determined by measuring the level of RNA products. For example, one could use nucleic acid sequences that hybridize to a RNA product of the TRPM1 biomarker.

The term “isolated nucleic acid sequence” as used herein refers to a nucleic acid substantially free of cellular material or culture medium when produced by recombinant DNA techniques, or chemical precursors, or other chemicals when chemically synthesized. An “isolated nucleic acid” is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded. The nucleic acid sequences contemplated by the present application include isolated nucleotide sequences which hybridize to a RNA product of the biomarker of the present application, nucleotide sequences which are complementary to the RNA product of a biomarker of the present application, nucleotide sequences which act as probes, or nucleotide sequences which are sets of TRPM1 specific primers.

The term “hybridize” refers to the sequence specific non-covalent binding interaction with a complementary nucleic acid. In one embodiment, the hybridization is under stringent hybridization conditions. In anther embodiment, the hybridization is under moderately stringent conditions.

By “hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log10[Na+])+0.41(%(G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5× Denhardt's solution/1.0% SDS at Tm-5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol.3.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand, is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art. The term “TRPM1 specific primers” as used herein refers to a set of primers which can produce a double stranded nucleic acid product complementary to a portion of TRPM1 RNA product or sequences complementary thereof. In a specific embodiment, the TRPM1 specific primers have the nucleic acid sequence of SEQ ID NOS: 5 and 6.

The term “probe” as used herein refers to a nucleic acid sequence that will hybridize to a nucleic acid target sequence. In one example, the probe hybridizes to a TRPM1 RNA product or a nucleic acid sequence complementary to the TRPM1 RNA product. The length of probe depends on the hybridize conditions and the sequences of the probe and nucleic acid target sequence. In one embodiment, the probe is at least 8, 10, 15, 20, 25, 50, 75, 100, 150, 200, 250, 400, 500 or more nucleotides in length. In a specific embodiment, the TRPM1 probe has the nucleic acid sequence of SEQ ID NO: 7.

A person skilled in the art will appreciate that a number of methods can be used to measure or detect the level of RNA products of the biomarker of the present application within a sample, including microarrays, RT-PCR (including quantitative RT-PCR), nuclease protection assays and northern blots.

In addition to determining an RNA product, the level of biomarker product is optionally determined using a binding agent that specifically binds a biomarker protein product. Accordingly, in one embodiment, the method uses binding agents such as an isolated protein that binds protein products of the biomarker described in the present application.

The term “isolated protein” as used herein refers to a proteinaceous agent, such as a peptide, polypeptide or protein, which is substantially free of cellular material or culture medium when produced recombinantly, or chemical precursors, or other chemicals, when chemically synthesized.

The phrase “binds a protein product” as used herein refers to a binding agent such as an isolated protein that specifically binds a protein product of a particular biomarker described in the present application. The protein product bound is optionally a full-length biomarker protein product, or a fragment that is cleaved, secreted, released or shed from a cell. The protein product determined is optionally intracellular, extracellular or a combination thereof.

In one embodiment, the isolated protein that binds a biomarker protein product is an antibody or antibody fragment.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies having specificity for a specific protein, such as a protein product of a biomarker described in the present application, may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody-producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

In one embodiment, the binding agents, including isolated proteins or antibodies, are labeled with a detectable marker. The label is preferably capable of producing, either directly or indirectly, a detectable signal. For example, the label may be radio-opaque or a radioisotope, such as ³_{H,} ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In another embodiment, the detectable signal is detectable indirectly. For example, a secondary antibody that is specific for a biomarker described in the present application and contains a detectable label can be used to detect the biomarker.

The present application also contemplates the use of “peptide mimetics” for detecting TRPM1 biomarker protein products. Peptide mimetics are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of binding agents specific for protein products of the biomarkers described in the present application. Peptide mimetics also include peptoids, oligopeptoids (Simon et al (1972) Proc. Natl. Acad, Sci USA 89:9367).

A person skilled in the art will appreciate that a number of methods can be used to determine the amount of the protein product of the biomarker of the present application, including immunoassays such as Western blots, ELISA, and immunoprecipitation followed by SDS-PAGE immunocytochemistry.

### (v) Agents to Detect Single Nucleotide Polymorphisms

The detection of a single nucleotide polymorphism disclosed herein is optionally determined by detecting binding of a nucleic acid sequence that specifically hybridizes to the associated allele. Accordingly, in one embodiment, the present application provides a probe that specifically hybridizes to at least one of the SNP alleles associated with CSNB. In one embodiment, the at least one SNP allele associated with CSNB is an allele listed in Table 9. In another embodiment, the at least one SNP allele associated with CSNB is located at position 108249293 of ECA1, wherein the associated allele is an A nucleotide. In another embodiment, the present application provides a probe that specifically hybridizes to at least one of the SNP alleles associated with LP. In one embodiment, the at least one SNP allele associated with LP is an allele listed in Table 8. In another embodiment, the at least one SNP allele associated with LP is located at position 108249293 of ECA1, wherein the associated allele is an A nucleotide.

The term “a probe that specifically hybridizes to the associated allele” as used herein refers to a nucleic acid that binds to a sequence comprising the SNP associated allele and not to a sequence having a different nucleotide at the SNP position. For example, for the SNP at position 108370091 of ECA1, the probe that specifically hybridizes to the associated allele would be a probe that binds a sequence that contains the T nucleotide but not a sequence that contains a different allele at the same position. For the SNP at position 108249293 of ECA1, the probe that specifically hybridizes to the associated allele would be a probe that binds a sequence that contains the A nucleotide but not a sequence that contains a different allele, such as a G nucleotide, at the same position.

In another embodiment, the detection of a single nucleotide polymorphism disclosed herein is optionally determined by analyzing, for example, by sequencing, the region comprising the SNP. In one embodiment, two or more isolated nucleic acid sequences that are specific primers are able to amplify the sequence containing the SNP. Pairs of primers may be selected wherein one primer is upstream of the SNP location and one primer is downstream of the SNP location. For example, for the SNP found at position 108370091 of ECA1, one primer is upstream of the nucleotide at position 108370091 and another primer is downstream from the nucleotide at position 108370091. For the SNP found at position 108370150 of ECA1, one primer is upstream of the nucleotide at position 108370150 and another primer is downstream from the nucleotide at position 108370150. For the SNP found at position 108249293, one primer is upstream of the nucleotide at position 108249293 and another primer is downstream from the nucleotide at position 108249293.

### (C) Kits

Another aspect of the present application is a kit for screening, detecting, or diagnosing congenital stationary night blindness in a subject or detecting or screening horse coat patterns. In one embodiment, the kit comprises an antibody to a TRPM1 protein product and instructions for use. In another embodiment, the kit comprises a TRPM1 probe or TRPM1 specific primers and instructions for use. In another embodiment, the kit comprises a probe that specifically hybridizes to a SNP allele as disclosed herein or specific primers that amplify a region comprising a SNP allele as disclosed herein and/or instructions for use. The kit can also include ancillary agents. For example, the kits can include vessels for storing or transporting the antibodies, probes and/or primers; a control; instruments for obtaining a sample; and/or buffers or stabilizers.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

To determine if differential expression could be the cause of LP and CSNB, the relative expression of candidate genes were evaluated by quantitative real-time RT-PCR. A local regulatory phenomenon will be ruled out by measuring the expression of three additional nearby genes. These included two genes positioned on either side of TRPM1—the OTU domain containing 7A (OTUD7A), and the myotubularin-related protein 10 (MTMR10)) and one gene more distal—tight junction protein 1 (TJP1)—according to the first assembly of the equine genome (http://www.genome.ucsc.edu/cgi-bin/hgGateway?org=Horse&db=equCab1) (FIG. 3).

### Materials and Methods

Horses and genotype categories: Horses were categorized according to genotype and phenotype for LP, which was diagnosed by coat color assessment, breeding records, and, for those horses used in the retinal study, also by ocular examination, including scotopic ERG. Horses were included in the LP/LP group if they had a “fewspot” or “snowcap blanket” pattern and a scotopic ERG consistent with CSNB (FIG. 1a). Horses in the LP/lp group all displayed white patterning with dark spots and/or had breeding records consistent with heterozygosity (“leopard,” “spotted blanket,” or “lace blanket” patterns) and a normal scotopic ERG. Horses were included in the non-appaloosa (lp/lp) group if they were solid colored and showed no other traits associated with the presence of LP (striped hooves, white sclera, and mottled skin) and a normal scotopic ERG. The non-appaloosa horses were from the Thoroughbred and American Quarter Horse breeds, two breeds that are not known to possess any appaloosa spotted individuals. Due to the invasive nature of some of the experiments performed, it was impossible to obtain a significant number of samples from age, sex, and base-coat-color-matched horses. Both male and female horses were used in this study, horses ranged in age from <1 year to 23 years old, and the base coat colors of black, bay, and chestnut were all represented (Table 1).

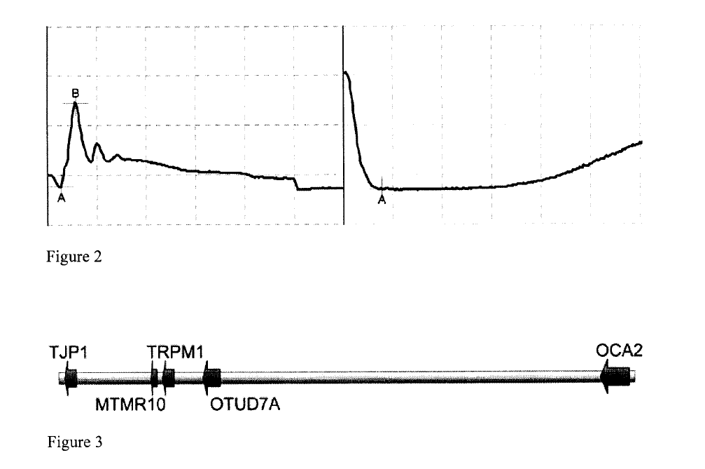

Ophthalmic Examinations: Horses used in this study were categorized by ocular examination, which included neurophthalmic examination, slit-lamp biomicroscopy (SL-14, Kowa, Japan), indirect ophthalmoscopy (Heine Omega 200, Heine Instruments), and electroretinography (Cadwell Sierra II, Cadwell Laboratories, Kenewick, Wash.). For electroretinography, horses were sedated with 10 μg/kg detomidine hychloride (Dormosedan, Orion Pharma, Pfizer Animal Health, Kirkland, QC, Canada) by intravenous bolus. Pharmacological mydriasis was achieved with 0.2 ml 1% tropicamide (1% mydriacyl, Alcon, Mississauga, ON, Canada). Auriculopalpebral nerve blocks were performed using 2 ml of a 2% lidocaine hychloride injectable solution (Bimeda-MTC Animal Health, Cambridge, ON, Canada). Scotopic ERGs were completed bilaterally to identify nyctalopia and CSNB. A corneal DTL microfiber electrode (DTL Plus Electrode, Diagnosys, Littleton, Mass.) was placed on the cornea, and platinum subdermal needle electrodes (Cadwell Low Profile Needle electrodes, Cadwell Laboratories) were used as reference and ground. The reference electrode was placed subdermally 3 cm from the lateral canthus and the ground electrode was placed subdermally over the occipital bone. The ERGs were elicited with a white xenon strobe light and recorded with a Cadwell Sierra II (Cadwell Laboratories) with the bandwidth set at 0.3-500 Hz; eyelids were held open manually for each test and a pseudo-Ganzfeld was used to attempt even stimulation of the entire retina (Komaromy et al. 2003). Horses were dark adapted for 25 min and dark-adapted ERG responses were stimulated using maximum light intensity with each recording representing the average of 20 responses. An a-wave dominated ERG or “negative ERG” was considered diagnostic of CSNB (Witzel et al. 1977; Sandmeyer et al. 2007). Horses included in the LP/LP (n=4) group had a “negative ERG,” and those in the LP/lp group (n=4) and lp/lp group (n=6) had normal scotopic and phototopic electroretinograms (FIG. 2, Table 2).

Retina and collection and RNA isolation: Horses were humanely euthanized by intravenous overdose of barbiturate (Euthanyl, MTC Pharmaceuticals) following the Canadian Council on Animal Care Guidelines for Experimental Animal Use and approved by the University of

Saskatchewan Animal Care Committee. The eyes were removed immediately and placed on ice. The posterior segment of the globes were isolated by removing the anterior segment via a 360° incision posterior to the limbus. The vitreous was removed by gentle traction. In one eye from each horse, the retina was detached from the periphery and was transected at the optic nerve with Vannas scissors. For the second eye from each horse, the posterior segment was transected with a scalpel blade and one-half was prepared for histology. The retina was removed from the remaining posterior segment and added to the entire retina of the first eye. Retina was then centrifuged and suspended in the appropriate volume of Trizol (Invitrogen) and homogenized in a Polytron mechanical homogenizer (Brinkman Instruments, Westbury, N.Y.). Total retinal RNA was isolated according to the manufacturer's instructions and stored at −80° until used.

Skin collection and RNA Isolation: Skin samples from seven homozygous appaloosa spotted horses (LP/LP), seven heterozygotes (LP/lp), and seven non-appaloosa (lp/lp) were obtained. Samples were taken from live horses (with appropriate consent of owner) and from those euthanized as described above. Donor skin sites of the live horses were infiltrated with a local anesthetic (2% lidocaine hychloride, Bimeda-MTC Animal Health, Cambridge, ON, Canada). Following hair removal by shaving the sample area, five 6-mm dermal punch biopsies were collected and immediately snap frozen in liquid nitrogen. Samples were placed at -80° until processing. From each horse in the LP/LP group and LP/lp group, two sample areas were collected for RNA extraction: one sample area that was pigmented (i.e., a darkly pigmented body spot) and one area where skin and hair where completely unpigmented. Skin samples from euthanized horses were collected in a similar fashion; however, punch biopsies were not used. Instead 10×1-cm² sections of skin were harvested from each site by sharp incision with a sterile no. 22 scalpel blade (Paragon, Sheffield, England). A new scalpel blade and a new pair of sterile gloves were worn to perform the harvest from each site to avoid transfer of genetic material. Prior to RNA isolation, skin samples were first powdered by crushing under liquid nitrogen. Total RNA was isolated from 0.5 g of tissue in a buffer of 4 M guanidinium isothiocyanate, 0.1 M Tris-HCl, 25 mM EDTA (pH 7.5), and 1% (v/v) 2-mercaptoethanol, followed by differential alcohol and salt precipitations (Chomczynski and Sacchi 1987; MacLeod et al. 1996). All samples were stored at −80°.

Quantitative real-time RT-PCR: RNA was quantified using a NanoDrop spectrophotometer (NanoDrop Technologies, Wilmington, Del.) and sample concentrations were adjusted to 50 ng/ml with RNAse free water (Ambion, Austin, Tex.). RNA integrity and purity was verified using a Bioanalyzer (Agilent Technologies, Santa Clara, Calif.). All skin and retinal samples isolated were of high purity and integrity, and all samples used had RNA integrity numbers >8, limiting the RPE sample group to n=4 for the (LP/LP) sample group, n=3 for the (LP/lp) sample group and n=4 for the (lp/lp) control group.

Equine homologs for TRPM1, OCA2, TJP1, MTMR10, and OTUD7A were identified from the Entrez Trace Archive using a Discontiguous Megablast (http://www.ncbi.nih.gov/BLAST) or by a BLAT search against the horse (January 2007) (equCab I) assembly (http://www.genome.ucsc.edu/). Taqman primers and probes were designed as previously described (Murphy et al. 2006). Preliminary experiments showed that) β-Actin was the most stable reference gene among those tested in the samples. The PCR efficiency of primer/probe combinations were calculated using serial dilutions of RNA spanning a magnitude of eightfold (or greater) by the REST analysis program (Pfaffl et al. 2002). R² values for standard curves were ≧0.98 for all products tested (Table 3). All primer pairs were tested to ensure that genomic DNA was not being amplified by using a minus reverse transcription control in each assay.

Taqman quantitative real-time RT-PCR was performed using a Smart Cycler real-time thermal cycler (Cepheid, Sunnyvale, Calif.). Each 25 μl reaction contained 250 ng of RNA, 1×EZ buffer (Applied Biosystems, Foster City, Calif.), 300 μM of each dNTP, 2.5 mM manganese acetate, 200 nM forward and reverse primer, 125 nM fluorogenic probe, 40 U RNasin (Roche, Indianapolis, Ind.) and 2.5 U rTth (Applied Biosystems). Cepheid also recommends the addition of an ‘additive reagent’ to prevent binding of polymerases and nucleic acids to the reaction tubes. This reagent was added to give a final concentration of 0.2 mg/ml bovine serum albumin (non-acetylated), 0.15 M trehalose and 0.2% Tween 20. Thermocycler parameters for all assays consisted of a 30-min reverse transcription (RT) step at 60° C., 2 min at 94° C. and 45 cycles of: 94° C. for 15 s (denaturation) and 60° C. for 30 s (annealing and extension). The threshold crossing cycle (Cₜ) values generated by the Smart Cycler were used to calculate the relative expression ratios and statistical significance between each group of horses for each tissue tested using REST-MCS version-2. The relative mean expression ratios were calculated according to the following mathematical model: relative expression ratio (R)=(E_{target})^{ΔCt(target)}/(E_{reference})^{ΔCt(reference) (Pfaffl} 2001), where E represents the calculated efficiencies for the corresponding genes, Cₜ is the crossing threshold cycle number, and ΔCₜ(target) and ΔCₜ(reference) represent the Cₜ difference between the control group (non-appaloosa horses lp/lp) and the experimental group (either LP/LP or LP/lp) for the target and the reference (β-actin) transcripts, respectively. Given the variability that may occur among individual samples, REST was used to analyze the data to make group-wise comparisons within the populations. REST makes no assumptions about the distribution of observations in the population and thus has been shown to be an appropriate statistical model for analyzing gene expression population data (Pfaffl et al. 2002). This gene expression software tool calculates mean expression ratios for each of the sample groups being tested and then runs permutation tests to determine if the results are due to random allocation or to the effects of treatment (which in this case is the genotype at the LP locus). Gene expression was analyzed with the pairwise fixed reallocation randomization test using REST software to compare gene expression of homozygotes (LP/LP) and heterozygotes (LP/lp) relative to non-appaloosa skin (lp/lp) and to compare CSNB affected (LP/LP) and CSNB unaffected (LP/lp) relative to unaffected (lp/lp) retina. Data are expressed as both relative expression ratios (R) and as foldchanges (FC). Data are log transformed for graphical representation so that large relative expression differences can be easily visualized on a graph. See Tables 4, 5, 6 and 7.

### Results and Discussion

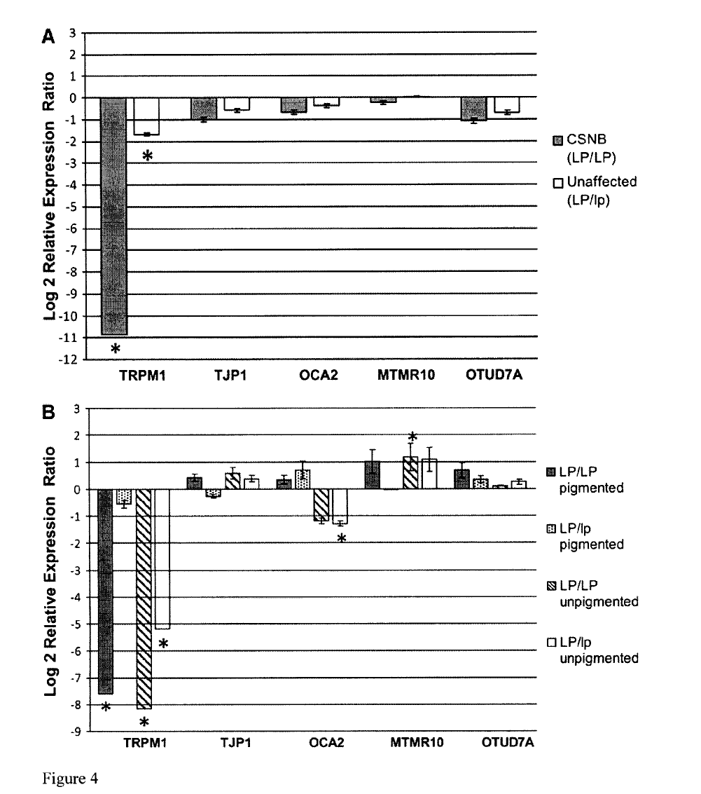

TRPM1 as the gene for CSNB in Appaloosa horses: TRPM1 was the only gene of those investigated that was differentially expressed in the retina. In the retina of CSNB (LP/LP) horses, expression was 0.05% of the level found in non-appaloosa horses (R=0.0005). This constitutes an FC decrease >1800. (FC=−1870.637, P=0.001). TRPM1 was marginally downregulated in horses heterozygous for appaloosa spotting (LP/lp) (R=0.312, FC=−3.201, P=0.005) (FIG. 4A; Table 4). It is possible that the downregulation of TRPM1 in the retina of LP/LP horses is the etiology of CSNB. TRPM1 may play a role in neural transmission in the retina through changing cytosolic free Ca²⁺ levels in the retinal ON bipolar cells. The MGIuR6 receptors of the ON bipolar cells are coupled to Gαo proteins, the most abundant heteromeric G protein in the brain. However, there are no known downstream targets of Gαo proteins (Duvoisin et al. 2005). These observations lead to speculation that TRPM1 is a cation channel that is a downstream target of the Gαo protein in the ON bipolar cell. In dark adaptation, the cation channel activity of TRPM1 would be turned off by glutamate binding to the MGIuR6 receptor. Light-induced decreases in synaptic glutamate concentration could remove a negative Gαo signal from TRPM1, leading to cation currents that depolarize the ON bipolar cell. Most recently, expression of TRPM1 has been detected specifically in retinal bipolar cells, further supporting the possibility that lack of TRPM1 is responsible for the failure of b-wave perpetuation (Koike et al. 2007).

Alterations in TRPM1 may cause appaloosa spotting: Compared to skin from non-appaloosa horses (lp/lp), TRPM1 was significantly downregulated (P=0.001) in both pigmented (R=0.005, FC=−193.963, P=0.001) and unpigmented (R=0.003, FC=−288.686, P=0.001) skin from homozygous (LP/LP) horses. In unpigmented skin from heterozygous (LP/lp) horses, TRPM1 was downregulated to a lesser extent (R=0.027, FC=−36.583, P=0.001) (FIG. 4B, Table 5). However, gene expression values for heterozygotes were not half the difference between appaloosa homozygotes and non-appaloosa horses, indicating that the difference is not a simple dosage effect. Relative expression differences at or near this magnitude were not detected for any of the other genes tested from this chromosome region (FIG. 4B). When compared to mRNA from non-appaloosa skin samples, small changes with less stringent P-values were detected for OCA2 and MTMR10 in LP/lp and LP/LP unpigmented skin samples, respectively (Table 5). These small changes are likely due to the generalized difference between pigmented and unpigmented skin rather than a direct effect of LP.

In humans TRPM1 is expressed in several isoforms (Xu et al. 2001: Fang and Setaluri 2000). The long isoform, termed MLSN-L, is thought to be responsible for Ca²⁺ influx (Xu et al. 2001). Primers and probes were designed to specifically detect this long isoform. It is possible the large relative expression difference that was detected for the long isoform of TRPM1 may interfere with Ca²⁺ signaling in the melanocytes and thus participate in the biological mechanisms of appaloosa spotting.

The specific function of TRPM1 in melanocytes is unknown. It has been described as a tumor suppressor that may regulate the metastatic potential of melanomas, as its expression declines with increased metastatic potential (Duncan et al. 1998; Deeds et al. 2000; Duncan et al. 2001). Treatment of pigmented melanoma cells with a differentiation inducing agent upregulated the long isoform of this gene (Fang and Setaluri, 2000). TRPM1 therefore has potential roles in Ca²⁺-dependent signaling related to melanocyte proliferation, differentiation, and/or survival.

One potential role of TRPM1 in melanocyte survival is in interaction with the signaling pathway of the cell surface tyrosine kinase receptor, KIT, and its ligand KITLG. Signaling through the KIT receptor is critical for the growth, survival and migration of melanocyte precursors (reviewed by Erikson, 1993). It has been shown that both phospholipase C activation and Ca²⁺ influx are important in supporting KIT-positive cells (Berger 2006). Stimulation with KIT ligand while blocking Ca²⁺ influx led to a novel form of cell death that is termed activation enhanced cell death (AECD) (Gommerman and Berger 1998). It is possible that during melanocyte proliferation and differentiation, when KIT positive cells are being stimulated by the ligand in vivo, the absence of TRPM1 expression may result in decreased Ca²⁺ influx and ultimately result in AECD. Early melanocyte death could explain LP hypopigmentation patterns.

Notably, TRPM1 expression in pigmented skin from heterozygous (LP/lp) horses did not differ significantly from that of non-appaloosa horses. TRPM1 expression is likely tissue specific as 4000 times greater expression was found in the retina than in skin (P=0.001). Similarly, temporal regulatory elements may direct relatively higher expression in migrating melanocyte precursors than in mature melanocytes; thus in the skin expression at the biological relevant time point may not have been measured. We have also shown an association between decreased TRPM1 expression and unpigmented LP/lp skin. However, further work is required to rule out the possibility that decreased expression of TRPM1 in unpigmented LP/lp skin when compared to non-appaloosa skin may simply reflect an absence of TRPM1-expressing melanocytes.

Summary and prospects: LP has been mapped to a 6-cM region on ECA1 containing the candidate genes TRPM1 and OCA2 (Terry et al. 2004; Bellone et al. 2006a). In addition, CSNB has been associated with homozygosity for LP (Sandmeyer et al. 2007). Herein it is reported that TRPM1 is the only gene from this candidate region that is significantly downregulated in the retina and skin of LP/LP horses. The previously published mapping data, in connection with this reported gene expression data, support the hypothesis that TRPM1 is the molecular mechanism for both LP and CSNB.

TRPM1 is significantly downregulated in the skin, retina, and RPE of (LP/LP) horses. These data represent the first report describing a gene expressional mechanism associated with an eye disease and coat color phenotype in the horse. Coding and regulatory regions will be investigated by sequencing analysis to identify the basis of the TRPM1 expressional loss observed. As was mentioned previously, three E-boxes and one M-box have been identified in the proximal promoter of this gene in humans and mouse. The newly available assembled equine genome will be used to identify regions of interest to investigate regions of interest for evidence of mutations in these regulatory elements. Many of the genes involved in melanogenesis have distinct distal regulatory elements that control their expression. For example, TYR has a distal regulatory element specific to melanocyte 15 kB away from the start of transcription (Porter et al. 1991; Ganss et al., 1994; Porter and Meyer, 1994). Novel distal regulatory elements of TRPM1 are likely to be identified. Appaloosa spotted horses may serve as an important research tool illustrating the role of TRPM1 in normal night vision and melanogenesis. Although several mutations have been identified as the cause of CSNB in humans (Dryja et al 2005; Zeitz et al. 2006; Xiao et al. 2006; Szabo et al. 2007) none of these mutations involve TRPM1. Thus, the horse could serve as a model for as-yet-unsolved forms of heritable human CSNB. In addition, mutations in CABP4, a member of the calcium binding protein family, were recently shown to cause a 30-40% reduction in transcript levels and result in an autosomal recessive form of CSNB in humans (Zeitz et al. 2006). Therefore studying the molecular interaction of TRPM1 and other genes causing CSNB involved in calcium signaling could lead to a better understanding of signal transduction during night vision.

### Example 2

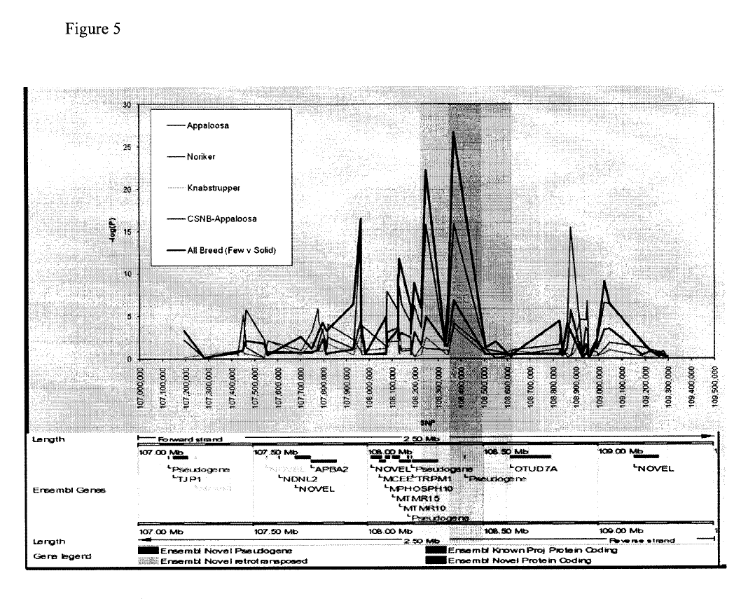

In order to confirm candidate gene causality and identify a potential region for further sequencing investigation, LP and CSNB were fine mapped by SNP analysis (FIG. 5). 70 SNPs spanning over 2 Mb were genotyped in 192 horses from three different breeds segregating for LP.

### Material and Methods

Genomic DNA was isolated from either whole blood using the PUREGENE DNA Isolation Kit (Gentra Systems Minneapolis, Minn., USA) or from hair follicles (Locke et al., 2002) from 192 horses from three different breeds; Appaloosa (N=127) Knabstrupper (N=29), and Noriker (N=36).

Thirty of these horses were examined by ERG for CSNB (14 case samples and 16 controls). LP genotype was determined by breeding records and LP spotting pattern phenotype.

Gene expression data implicated TRPM1 as the cause for both LP and CSNB therefore SNPs flanking this gene that spanned over 2 MB were analyzed (ECA1:107,194,138-109,299,508).

70 SNPs previously identified in the 2007 (equCab2) assembly (http://www.ensembl.org/Equus_caballus/index.html) of the horse genome were genotyped in all 192 horses by Sequenom Mass Spectrometry platform using the IPlex system.

SNPs were analyzed for association with LP genotype by Chi-Square analysis.

### Results

Horses homozygous for LP (LP/LP, cases) were compared to solid non-characteristic horses (lp/lp, controls). Only those SNPs with statistically significant results (P≦0.05) are presented in Table 8. SNPs 32-40 are in the region of high association and SNP 37 had the highest association and is represented by bold text.

Horses that were diagnosed with CSNB (cases) were compared to those that were unaffected (controls) as diagnosed by ERG. Only those SNPs with statistically significant results (P≦0.05) are presented in Table 9. The two SNPs with the highest association are bolded.

When comparing homozygous patterns (N=64, LP/LP) to solid (N=50, lp/lp) the strongest associated SNP for all breeds tested occurred at position ECA1:108370091 (X²=117.43, P=2.31×10⁻²⁷). (Table 8, FIG. 5). This corresponds to a region upstream of the coding region for TRPM1. TRPM1 is down regulated in CSNB and LP/LP horses, thus this region may contain important regulatory elements for this gene.

When comparing CSNB affected horses (N=13) versus controls (N=14) the strongest association occurred at positions ECA1:108370091 and ECA1: 108370150 (X²=27.53, P=1.55×10⁻⁰⁷) (Table 9, FIG. 5). This provided further support that LP and CSNB are strongly associated and perhaps caused by the same mutation.

These data have allowed refinement of the map position of LP from a region that was approximately 6 Mb to one that is approximately 300 Kb. These data further support TRPM1 as being the cause for LP and CSNB.

A 400 Kb region, including the region of highest association, are currently being investigated by hybrid selection and Solexa Sequencing analysis. Overall, the strongest association between LP, CSNB, and SNP genotype in all breeds tested occurred at position ECA1: 108370091 (P=2.31×10⁻²⁷).

### Example 3

### Identification of a SNP in TRPM1 and its Association with Leopard Complex Spotting (LP) and Congenital Stationary Night Blindness (CSNB) in Horses

### Materials and Methods

Primers to amplify and sequence regulatory regions, exons and flanking introns of TRPM1 were designed based on the publicly available Equine Genome sequence. (http://blast.ncbi.nlm.nih.gov/Blast.cgi?PAGE=Nucleotides&PROGRAM=blast n&BLAST_SPEC=TraceArchive&BLAST_PROGRAMS=meqaBlast&PAGETY PE=BlastSearch; http://www.qenome.ucsc.edu/cgi-bin/hqBlat?command=start).

Homo sapiens TRPM1 transcript (Refseq: NM 002420, Ensembl transcript ID ENST00000256552) was used to identify equine homologous sequence by BLAST or BLAT searches. (http://blast.ncbi.nlm.nih.qov/Blast.cqi?PAGE=Nucleotides&PROGRAM=blast n&BLAST_SPEC=TraceArchive&BLAST_PROGRAMS=megaBlast&PAGETY PE=BlastSearch; http://www.genome.ucsc.edu/cgi-bin/hqBlat?command=start; http://www.ensembl.org/Homo_sapiens/exonview?db=core;transcript=ENST0 0000256552).

Homo sapiens TRPM1 proximal promoter was used to identify the equine promoter from the Equine Trace archives by performing a BLAST search. http://blast.ncbi.nlm.nih.gov/Blast.cgi?PAGE=Nucleotides&PROGRAM=blastn &BLAST_SPEC=TraceArchive&BLAST_PROGRAMS=megaBlast&PAGETYP E=BlastSearch; and Zhiqi et al. (2004) Melanoma Res 14:509-516.)

Homo sapiens miRNA within TRPM1 (Accession : MI0000287 ID: hsa-mir-211) was used to identify homologous miRNA sequence in horse. (http://blast.ncbi.nlm.nih.gov/Blast.cgi?PAGE=Nucleotides&PROGRAM=blast n&BLAST_SPEC=TraceArchive&BLAST_PROGRAMS=megaBlast&PAGETY PE=BlastSearch; Griffiths-Jones S. et al. (2008) and Lim et al. (2003)).

Equine Exon 3-27 sequence was verified by cDNA sequencing from RNA isolated from retina.

DNA from one solid non-Appaloosa (lp/lp) and one homozygous appaloosa (LP/LP) was amplified using standard PCR procedures.

Amplicons were gel purified using QIAquick gel extraction kit (Qiagen Sciences, Md., USA) and subsequently sequenced using BigDye® Terminator v1.1 and ABI 310 Genetic Analyzer (Applied Biosystems, Foster City, Calif. USA) or sent to the Core Sequencing Facility at the Plant Biotechnology Institute of the National Research Council, SK, Canada.

Sequences were analyzed by aligning the LP/LP and lp/lp sequence data using ContigExpress from the Vector NTI Advance 10.3 software package (Invitrogen Corporation, Carlsbad, Calif.). SNPs were identified by comparing these sequences with that of EquCab2 assembly (http://www.ensembl.org/Equus_caballus/index.html).

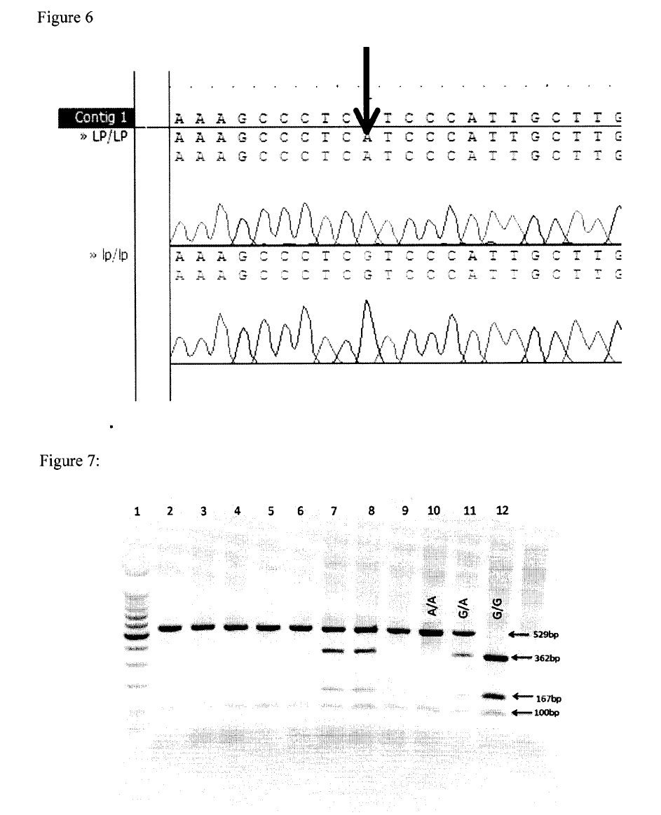

A SNP detected from the LP/LP sample in intron 11-12 was verified by sequencing a panel of 10 horses from different breeds. (FIG. 6)

The association of this SNP with LP and CSNB was tested by either direct sequencing or by a BsmFI PCR-RFLP. (FIGS. 6 and 7) All horses used for the CSNB analysis were examined and diagnosed by ERG (Sandmeyer L. et al. (2007)). A total of 357 horses from three different breeds known to have leopard complex spotting were tested. As a control group 32 Thoroughbreds, not expected to have the LP gene, were also tested.

### Results and Discussion

31 regions of TRPM1 spanning 13,067 bp were sequenced and 18 SNPs identified; 7 of which were detected in the solid non-appaloosa sample (lp/lp), 7 were detected in both the LP/LP and lp/lp sequence, and 4 detected only in the LP/LP sample when compared to the EquCab2. All 4 SNPs detected in the LP/LP sample were detected within an intron.

The 4 SNPs detected in the LP/LP sample were detected in:

amplified regions of exon 4 and flanking introns (SNP at 108267503 of ECA1 T>C, intron, forward primer: 5′-TCCAAAGTTCCCTTCCATCA-3′ (SEQ ID NO:20), reverse primer: 5′-TGCCAGAATGTTGACCATGT-3′ (SEQ ID NO:21));

exon 11 and flanking introns (SNP at 108249293 G>A, intron, forward primer: 5′-GACTGAGCGTATGTGCGTGT-3′ (SEQ ID NO:22), reverse primer: 5′-CTCCTGTCTGAGTGGCTTCA-3′ (SEQ ID NO:23)); and

exon 13 and flanking regions (SNP at 108246967 T>C, intron and SNP at position 108247024 T>C, intron, both amplified with forward primer: 5′-TAACCATGACCAGTCCTATC-3′ (SEQ ID NO:24), reverse primer: 5′-GCACCAGTCTATCATGTGTG-3′ (SEQ ID NO:25)).

One SNP (LP/LP: 108249293 G>A) showed perfect concordance with the LP genotype in a panel of ten horses. The LP genotype was predicted by phenotype from direct observation or photographic record.

There was a complete association with homozygosity of this SNP and CSNB (X²=30 p<0.0005). In addition there was a complete association for this SNP and LP genotype as for the Appaloosa (X²=298, p<0.0005) and the Knabstrupper horses (X²=68 p<0.0005). Among the Norikers, a very strong association was observed (X²=82.49, p<0.0005) however this association was not complete, 34 horses not suspected to have an LP allele possessed at least one copy of this SNP (Table 10).

Predicting genotype of LP from phenotype can be difficult because of the variability in pattern and complications when other spotting patterns caused by different loci are present.

In summary, one SNP, found within an intron, had a distribution identical to LP on a panel of 10 horses. To determine the strength of the association, 389 unrelated horses from different breeds were tested. There was a complete association of this SNP with LP (P<0.0005) and CSNB (N=30, P<0.0005) among Appaloosa and Knabstrupper horses. Among Noriker horses, a strong association was observed (P<0.0005) however this association was not complete; 34 horses identified as non-patterned (lp/lp) possessed at least one copy of this SNP.

While the present disclosure has been described with reference to what are presently considered to be the preferred examples, it is to be understood that the application is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Base coat color, proposed LP genotype, disease status, age, sex
and tissue sampled for each horse used in qRT-PCR experiments.

| Horse Sample | | Proposed LP | CSNB | age at | | Tissue |
|---|---|---|---|---|---|---|
| number | Base color | genotype | phenotype | sampling | sex | sampled |
| 05-10 | bay dun | LP/LP | CSNB | 5 | mare | skin |
| 05-12 | black | LP/LP | CSNB | 13 | mare | skin |
| 05-13 | chestnut | LP/LP | CSNB | 5 | mare | skin |
| 06-261 | black | LP/LP | not examined | 15 | stallion | skin |
| 06-222 | bay | LP/LP | CSNB | 5 months | mare | skin |
| 07-51 | liver chestnut | LP/LP | CSNB | 4 | gelding | skin/retina |
| 07-54 | chestnut | LP/LP | CSNB | 1 | stallion | skin/retina |
| 07-53 | chestnut | LP/LP | CSNB | 1 | stallion | retina |
| 07-52 | chestnut | LP/LP | CSNB | 1 | stallion | retina |
| 05-14 | black | LP/lp | normal | 2 | stallion | skin |
| 05-15 | dark bay | LP/lp | not examined | 2 | stallion | skin |
| 05-18 | bay dun | LP/lp | normal | 5 | gelding | skin |
| 07-49 | chestnut | LP/lp | normal | unknown | gelding | skin/retina |
| 07-50 | bay | LP/lp | normal | 3 | gelding | skin/retina |
| 06-275 | chestnut | LP/lp | not examined | 11 | mare | skin |
| 06-268 | black | LP/lp | normal | 1 | gelding | skin/retina |
| 06-269 | bay dun | LP/lp | normal | 1 | gelding | retina |
| 05-48 | red dun | lp/lp | not examined | 3 | gelding | skin |
| 05-49 | dark bay | lp/lp | not examined | 23 | mare | skin |
| D052 | bay | lp/lp | not examined | 4 | stallion | skin |
| 06-270 | chestnut | lp/lp | normal | 6 months | stallion | skin |
| 06-271 | dark bay | lp/lp | normal | 7 | mare | skin/retina |
| 07-46 | chestnut | lp/lp | normal | 1 | stallion | skin/retina |
| 07-48 | bay | lp/lp | normal | 2 | mare | skin/retina |
| 07-47 | buckskin | lp/lp | normal | 1 | mare | retina |
| 07-44 | bay | lp/lp | normal | 17 | mare | retina |
| 07-45 | chestnut | lp/lp | normal | 1 | stallion | retina |

TABLE 2
Scotopic ERG results for sample horses used in retinal study.

| | LP/LP | LP/lp | lp/lp |
|---|---|---|---|
| Number | 4 | 4 | 6 |
| Normal Scotopic ERG | 0 | 4 | 6 |
| “Negative” Scotopic ERG | 4 | 0 | 0 |

TABLE 3
Primer and Probe sequences and PCR efficiency used in quantitative
real-time RT-PCR.

| | Primer/ | | | PCR | |
|---|---|---|---|---|---|
| Gene | Probe | Sequence | Exon | Efficiency | R2 |
| B-Actin | Forward | 5′-GCCGTCTTCCCCTCCAT-3′ | 2 | 2.07 | 1 |
| | | (SEQ ID NO: 2) | | | |
| | Reverse | 5′-GCCCACGTATGAGTCCTTCTG-3′ | 3 | | |
| | | (SEQ ID NO: 3) | | | |
| | Probe | 5′-GGCACCAGGGCGTGATGGTGGGC-3′ | 2 and 3 | | |
| | | (SEQ ID NO: 4) | | | |
| TRPM1 | Forward | 5′-GACGACATCTCCCAGGATCT-3′ | 16 | 2.09 | 0.99 |
| | | (SEQ ID NO: 5) | | | |
| | Reverse | 5′-TGCTCGTCGTGCTTATAGGA-3′ | 17 | | |
| | | (SEQ ID NO: 6) | | | |
| | Probe | 5′-ATTCAAAAGACTTTGGCCAGCTGGC-3′ | 16 and 17 | | |
| | | (SEQ ID NO: 7) | | | |
| OCA2 | Forward | 5′-AGATCAAGGAAAGTTCTGGCAGT-3′ | 6 | 2.19 | 0.99 |
| | | (SEQ ID NO: 8) | | | |
| | Reverse | 5′-CTGGAGCAGCGTGGAATC-3′ | 7 | | |
| | | (SEQ ID NO: 9) | | | |
| | Probe | 5′-AAGCTACTCTGTGAACCTCAGCAGCCAT-3′ | 6 and 7 | | |
| | | (SEQ ID NO: 10) | | | |
| TJP1 | Forward | 5′-ATATGGGAACAACACACAGTGA-3′ | 2 | 2.18 | 0.98 |
| | | (SEQ ID NO: 11) | | | |
| | Reverse | 5′-GGTCCTCCTTTCAGCACATC-3′ | 3 | | |
| | | (SEQ ID NO: 12) | | | |
| | Probe | 5′-CTTCACAGGGCTCCTGGATTTGGAT-3′ | 2 and 3 | | |
| | | (SEQ ID NO: 13) | | | |
| MTMR10 | Forward | 5′-TGTCAGATTTCGCTTTGATGA-3′ | 5 | 2.28 | 0.98 |
| | | (SEQ ID NO: 14) | | | |
| | Reverse | 5′-GGTCTGTTGGCTGGGAATAA-3′ | 6 | | |
| | | (SEQ ID NO: 15) | | | |
| | Probe | 5′-TCAGGTCCTGAAAGTGCCAAAAAGG-3′ | 5 and 6 | | |
| | | (SEQ ID NO: 16) | | | |
| OTUD7A | Forward | 5′-CAGACTTTGTTCGGTCCACA-3′ | 3 | 2.27 | 0.98 |
| | | (SEQ ID NO: 17) | | | |
| | Reverse | 5′-AGTCACTCAGAGCGGCTGTC-3′ | 4 | | |
| | | (SEQ ID NO: 18) | | | |
| | Probe | 5′-AGAACCTGGTCTGGCCAGAGACCTG-3′ | 4 | | |
| | | (SEQ ID NO: 19) | | | |

TABLE 4
Statistically significant results from qRT-PCR of retinal tissue samples
(normalized to B-actin) relative to expression for
non-appaloosa horses (lp/lp).
Only statistically significant loci are presented.

| | n (control, | TRPM1 | | |
|---|---|---|---|---|
| Sample Group | sample)^{a} | R^{b} = | Direction | Significance^{c} |
| CSNB (LP/LP) | 6, 4 | 0.0005 | Down | P = 0.001 |
| Normal (LP/lp) | 6, 4 | 0.312 | Down | P = 0.005 |

^{a}RNA isolated from lp/lp retina samples with normal night vision as diagnosed by ERGs were used as controls. Data are expressed relative to these controls.
^{b}R = Relative expression ratio
^{c}Statistically significant results (P ≦ 0.05).

TABLE 5
Statistically significant results from qRT-PCR of skin tissue samples (normalized to B-actin) relative
to expression for non-appaloosa horses (lp/lp). Only statistically significant loci are presented.

| | n | | | | | | | | | |
|---|---|---|---|---|---|---|---|---|---|---|
| Sample | (control, | TRPM1 | Direc- | Signif- | OCA2 | Direc- | Signif- | MTMR10 | Direc- | Signif- |
| group | sample)^{a} | R^{b} = | tion | icance^{c} | R^{b} = | tion | icance^{c} | R^{b} = | tion | icance^{c} |
| Pigmented | 7, 7 | 0.005 | Down | P = | 1.267 | Up | P = | 2.027 | Up | P = |
| LP/LP | | | | 0.001 | | | 0.591 | | | 0.078 |
| Pigmented | 7, 7 | 0.681 | Down | P = | 1.629 | Up | P = | 0.977 | Down | P = |
| Lp/lp | | | | 0.465 | | | 0.285 | | | 0.946 |
| Unpig- | 7, 7 | 0.003 | Down | P = | 0.436 | Down | P = | 2.267 | Up | P = |
| mented | | | | 0.001 | | | 0.090 | | | 0.031 |
| LP/LP | | | | | | | | | | |
| Unpig- | 7, 7 | 0.027 | Down | P = | 0.411 | Down | P = | 2.117 | Up | P = |
| mented | | | | 0.001 | | | 0.031 | | | 0.091 |
| Lp/lp | | | | | | | | | | |

^{a}RNA isolated from lp/lp skin samples were used as controls. Data are expressed as relative to these controls.
^{b}R = Relative expression ratio
^{c}Highlighted in bold are statistically significant results (P ≦ 0.05).

TABLE 6
Statistical analysis of qRT-PCR data for RPE samples normalized
to B-actin.

| | | TRPM1 | | |
|---|---|---|---|---|
| Sample | n (control, | Fold | | |
| Group | sample)^{a} | Change | Direction | Significance^{b} |
| CSNB | 4, 4 | 1272.038 | Down | 0.0015 |
| (LP/LP) | | | | |
| Normal | 4, 3 | 3.394 | Down | 0.1885 |
| (LP/lp) | | | | |

^{a}RNA isolated from lp/lp RPE samples with normal night vision as diagnosed by ERGs was used as control for comparison to both homozygous appaloosas (LP/LP) affected with CSNB and heterozygous unaffected appaloosas (LP/lp).
^{b}Highlighted in bold are statistically significant results (P ≦ 0.01).

TABLE 7
Comparison of pigmented skin to unpigmented skin:
analysis of qRT-PCR data normalized to B-actin.

| | OCA2 | | | OTUD7A | | | TRPM1 | |
|---|---|---|---|---|---|---|---|---|
| | Fold | Direc- | Signif- | Fold | Direc- | Signif- | Fold | Direc- |
| sample/control^{a} | Change | tion | icance^{b} | Change | tion | icance^{b} | Change | tion |
| (LP/lp) | 3.960 | Down | 0.001 | 1.065 | Down | 0.818 | 24.91 | Down |
| unpigmented/ | | | | | | | | |
| (LP/lp) pigmented | | | | | | | | |
| (LP/LP) | 2.907 | Down | 0.025 | 1.499 | Down | 0.262 | 1.489 | Down |
| unpigmented/ | | | | | | | | |
| (LP/LP) pigmented | | | | | | | | |

| | | MTMR10 | | | TJP1 | | |
|---|---|---|---|---|---|---|---|
| | Signif- | Fold | Direc- | Signif- | Fold | Direc- | Signif- |
| sample/control^{a} | icance^{b} | Change | tion | icance^{b} | Change | tion | icance^{b} |
| (LP/lp) | 0.001 | 2.166 | Up | 0.015 | 1.558 | Up | 0.068 |
| unpigmented/ | | | | | | | |
| (LP/lp) pigmented | | | | | | | |
| (LP/LP) | 0.5375 | 1.119 | Up | 0.7015 | 1.116 | Up | 0.7005 |
| unpigmented/ | | | | | | | |
| (LP/LP) pigmented | | | | | | | |

^{a}Within group comparisons between pigmented and unpigmented samples were performed for the homozygous appaloosa group (LP/LP) and the heterozygous group (Lp/lp). In each case the control group was the pigmented sample.
^{b}Highlighted in bold are statistically significant results (P ≦ 0.05).

TABLE 8
Association analysis of 70 SNPs on ECA1 and LP genotype
among Appaloosa, Knabstrupper, and Noriker horses

| SNP | SNP position on | Associated | Ratio of Major and Minor | Chi- | |
|---|---|---|---|---|---|
| Number | ECA1 | Allele | Alleles (Case, Control) | Square | Probability |
| 1 | 107194138 | C | 23:105, 3:95 | 12.116 | 0.0005 |
| 5 | 107464256 | A | 89:37, 50:44 | 7.042 | 0.008 |
| 6 | 107542173 | G | 105:9, 71:17 | 5.779 | 0.0162 |
| 8 | 107701604 | C | 128:0, 93:7 | 9.244 | 0.0024 |
| 12 | 107796380 | C | 122:2, 80:16 | 16.322 | 5.343E−05 |
| 14 | 107815200 | C | 128:0, 91:9 | 11.993 | 0.0005 |
| 16 | 107931910 | A | 124:2, 74:22 | 25.708 | 3.971E−07 |
| 17 | 107965305 | A | 121:7, 41:53 | 71.237 | 3.169E−17 |
| 18 | 107965422 | G | 123:5, 78:20 | 15.363 | 8.871E−05 |
| 20 | 108074144 | T | 126:0, 84:14 | 19.2 | 1.177E−05 |
| 21 | 108078468 | G | 83:45, 42:46 | 6.266 | 0.0123 |
| 22 | 108128461 | C | 83:43, 39:55 | 12.958 | 0.0003 |
| 23 | 108128561 | T | 122:0, 65:25 | 38.419 | 5.706E−10 |
| 24 | 108131916 | A | 128:0, 69:29 | 43.453 | 4.342E−11 |
| 25 | 108132170 | T | 126:0, 62:26 | 42.376 | 7.532E−11 |
| 26 | 108132263 | C | 128:0, 64:32 | 49.778 | 1.722E−12 |
| 28 | 108181934 | T | 127:1, 80:20 | 24.797 | 6.369E−07 |
| 29 | 108182386 | C | 126:0, 92:8 | 10.45 | 0.0012 |
| 30 | 108197355 | C | 125:1, 66:26 | 36.97 | 1.2E−09 |
| 31 | 108227501 | C | 128:0, 81:17 | 24.01 | 9.583E−07 |
| 32 | 108248113 | G | 128:0, 42:56 | 97.237 | 6.151E−23 |
| 34 | 108329772 | G | 127:1, 92:8 | 7.716 | 0.0055 |
| 35 | 108340357 | A | 120:0, 77:15 | 21.055 | 4.463E−06 |
| 36 | 108343655 | C | 128:0, 81:19 | 26.531 | 2.594E−07 |
| 37 | 108370091 | T | 128:0, 31:61 | 117.43 | 2.312E−27 |
| 39 | 108507271 | A | 128:0, 97:3 | 3.891 | 0.0485 |
| 40 | 108549650 | C | 128:0, 93:5 | 6.678 | 0.0098 |
| 43 | 108827565 | G | 119:5, 73:21 | 17.062 | 3.619E−05 |
| 44 | 108832497 | G | 128:0, 88:10 | 13.666 | 0.0002 |
| 47 | 108861525 | G | 119:5, 74:20 | 15.66 | 7.58E−05 |
| 57 | 108991827 | G | 122:4, 86:12 | 6.838 | 0.0089 |
| 58 | 108992653 | C | 124:4, 80:16 | 12.371 | 0.0004 |
| 59 | 108992843 | T | 121:1, 82:8 | 8.296 | 0.004 |
| 60 | 109023700 | G | 121:5, 59:33 | 37.599 | 8.689E−10 |
| 61 | 109045266 | G | 109:19, 52:44 | 26.061 | 3.308E−07 |

TABLE 9
Association analysis of 70 SNPs on ECA1 and CSNB.

| | | | Ratio of | | |
|---|---|---|---|---|---|
| | | | and Major | | |
| | | Asso- | Minor Alleles | | |
| SNP | | ciated | (Case, | Chi- | |
| Number | SNP Position | Allele | Control) | Square | Probability |
| 12 | 107796380 | C | 26:0, 19:7 | 8.089 | 0.0045 |
| 17 | 107965305 | A | 25:1, 11:15 | 17.694 | 2.5938E−05 |
| 18 | 107965422 | G | 25:1, 16:10 | 9.339 | 0.0022 |
| 21 | 108078468 | G | 24:2, 13:13 | 11.337 | 0.0008 |
| 22 | 108128461 | C | 25:1, 13:13 | 14.075 | 0.0002 |
| 23 | 108128561 | T | 24:0, 17:7 | 8.195 | 0.0042 |
| 24 | 108131916 | A | 26:0, 20:8 | 8.72 | 0.0031 |
| 25 | 108132170 | T | 26:0, 18:6 | 7.386 | 0.0066 |
| 26 | 108132263 | C | 26:0, 18:8 | 9.455 | 0.0021 |
| 27 | 108140867 | T | 25:1, 15:11 | 10.833 | 0.001 |
| 28 | 108181934 | T | 26:0, 20:8 | 8.72 | 0.0031 |
| 30 | 108197355 | C | 26:0, 18:10 | 11.396 | 0.0007 |
| 31 | 108227501 | C | 26:0, 23:5 | 5.117 | 0.0237 |
| 32 | 108248113 | G | 26:0, 13:15 | 19.286 | 1.1255E−05 |
| 35 | 108340357 | A | 24:0, 20:4 | 4.364 | 0.0367 |
| 36 | 108343655 | C | 26:0, 22:6 | 6.268 | 0.0123 |
| 37 | 108370091 | T | 26:0, 8:18 | 27.529 | 1.5472E−07 |
| 38 | 108370150 | C | 26:0, 8:18 | 27.529 | 1.5472E−07 |
| 48 | 108878149 | C | 23:3, 6:20 | 22.531 | 2.0681E−06 |
| 51 | 108942855 | T | 26:0, 23:5 | 5.117 | 0.0237 |
| 52 | 108947019 | C | 26:0, 24:4 | 4.011 | 0.0452 |
| 57 | 108991827 | G | 26:0, 23:5 | 5.117 | 0.0237 |
| 58 | 108992653 | C | 26:0, 20:6 | 6.783 | 0.0092 |
| 60 | 109023700 | G | 26:0, 16:10 | 12.381 | 0.0004 |
| 61 | 109045266 | G | 24:2, 11:13 | 12.836 | 0.0003 |
| 69 | 109297525 | T | 3:23, 3:25 | 0.009 | 0.9233 |
| 70 | 109299508 | C | 3:23, 3:25 | 0.009 | 0.9233 |

TABLE 10
Genotyping Data for 108249293 G > A SNP in Four Horse Breeds

| | Genotype for SNP | | |
|---|---|---|---|
| LP proposed | 108249293 G > A | | |
| Genotype | A/A | A/G | G/G |

Appaloosa (N = 146)

| | | | |
|---|---|---|---|
| LP/LP | 64 | 0 | 0 |
| LP/lp | 0 | 59 | 0 |
| lp/lp | 0 | 0 | 23 |
| CSNB (N = 30) | | | |
| CSNB | 14 | 0 | 0 |
| unaffected | 0 | 6 | 10 |

Knabstrupper (N = 34)

| | | | |
|---|---|---|---|
| LP/LP | 14 | 0 | 0 |
| LP/lp | 0 | 19 | 0 |
| lp/lp | 0 | 0 | 1 |

Noriker (N = 177)

| | | | |
|---|---|---|---|
| LP/LP | 0 | 0 | 0 |
| Lp/lp | 4 | 59 | 0 |
| lp/lp all | 5 | 29 | 80 |

Thoroughbred (N = 32)

| | | | |
|---|---|---|---|
| LP/LP | 0 | 0 | 0 |
| LP/lp | 0 | 0 | 0 |
| lp/lp | 0 | 0 | 32 |

### REFERENCES

Bellone, R., T. Lear, D. L. Adelson and E. Bailey, 2006a Comparative mapping of oculocutaneous albinism type II (OCA2), transient receptor potential cation channel, subfamilyMmember 1 (TRPM1) and two equine microsatellites, ASB08 and 1CA43, among four equid species by fluorescence in situ hybridization. Cytogenet. Genome Res. 114: 93A.
Bellone, R., S. Lawson, N. Hunter, S. Archer and E. Bailey, 2006b Analysis of a SNP in exon 7 of equine OCA2 and its exclusion as a cause for appaloosa spotting. Anim. Genet. 37: 525.
Bennett, D. C., and M. L. Lamoreux, 2003 The color loci of mice: a genetic century. Pigment Cell Res. 16: 333-344.
Berger, S. A., 2006 Signaling pathways influencing SLF and c-kit mediated survival and proliferation. Immunol. Res. 35: 1-12.
Brooks, S. A., and E. Bailey, 2005 Exon skipping in the KIT gene causes a Sabino spotting pattern in horses. Mamm. Genome 11: 893-899.
Brooks, S., T. L. Lear, D. Adelson and E. Bailey, 2007 A chromosome inversion near the KIT gene and the Tobiano spotting pattern in horses. Cytogenet. Genome Res. 119: 225-230.
Brunberg, E., L. Andersson, G. Cothran, K. Sandberg, S. Mikko et al., 2006 A missense mutation in PMEL17 is associated with the silver coat color in the horse. BMC Genet. 7: 46.
Chomczynski, P., and N. Sacchi, 1987 Single-step method of RNA isolation by acid guanidinium thiocyanate-phenol-chloroform extraction. Anal. Biochem. 162: 156-159.
Clapham, D. E., L. W. Runnels and C. Stru″bing, 2001 The TRP ion channel family. Nat. Rev. Neurosci. 2: 387-396.
Deeds, J., F. Cronin and L. M. Duncan, 2000 Patterns of melastatin mRNA expression in melanocytic tumors. Hum. Pathol. 31: 1346-1356.
Dryja, T. P., T. L. McGee, E. L. Berson, G. A. Fishman, M. A. Sandberg et al., 2005 Night blindness and abnormal cone electroretinogram ON responses in patients with mutations in the GRM6 gene encoding mGluR6. Proc. Natl. Acad. Sci. USA 102: 4884-4889.
Duffy, D. L., G. W. Montgomery, W. Chen, Z. Z. Zhao, L. Le et al., 2007 A three-single-nucleotide polymorphism haplotype in intron 1 of OCA2 explains most human eye-color variation. Am. J. Hum. Genet. 80: 241-252.
Duncan, L. M., J. Deeds, J. Hunter, J. Shao, L. M. Homgren et al., 1998 Down-regulation of the novel gene melanstatin correlates with potential for melanomametastasis. Cancer Res. 58: 1515-1520.
Duncan, L. M., J. Deeds, F. E. Cronin, M. Donovan, A. J. Sober et al., 2001 Melastatin expression and prognosis in cutaneous malignant melanoma. J. Clin. Oncol. 19: 568-576.
Duvoisin, R. M., C. W. Morgans and W. R. Taylor, 2005 The mGluR6 receptors in the retina: analysis of a unique G-protein signaling pathway. Cell Sci. Rev. 2: 225-243.
Eiberg, H., J. Troelsen, M. Nielsen, A. Mikkelsen, J. Mengel-From et al., 2008 Blue eye color in humans may be caused by a perfectly associated founder mutation in a regulatory element located in the HERC2 gene inhibiting OCA2 expression. Hum. Genet. 123: 177-187.
Erickson, C. A., 1993 From the crest to the periphery: control of pigment cell migration and lineage segregation. Pigment Cell Res. 6: 336-347.
Fang, D., and V. Setaluri, 2000 Expression and up-regulation of alternatively spliced transcripts of melastatin, a melanoma metastasis-related gene, in human melanoma cells. Biochem. Biophys. Res. Commun. 279: 53-61.
Ganss, R., L. Montoliu, A. P. Monaghan and G. Schutz, 1994 A cell-specific enhancer far upstream of the mouse tyrosinase gene confers high level and copy number-related expression in transgenic mice. EMBO J. 13: 3083-3093.
Gardner, J. M., Y. Nakatsu, Y. Gondo, S. Lee, M. F. Lyon et al., 1992 The mouse pink-eyed dilution gene: association with human Prader-Willi and Angelman syndromes. Science 257: 1121-1124.
Gommerman, J. L., and S. A. Berger, 1998 Protection from apoptosis by steel factor but not interleukin-3 is reversed through blockade of calcium influx. Blood 91: 1891-1900.
Griffiths-Jones S. et al. (2008) NAR 36:D154-D158
Haase, B., S. A. Brooks, A. Schlumbaum, P. Azor, E. Bailey et al., 2007 Allelic heterogeneity at the equine KIT locus in dominant white (W) horses. PLoS Genet. 3: e195.
Hunter, J. J., J. Shao, J. S. Smutko, B. J. Dussault, D. L. Nagle et al., 1998 Chromosomal localization and genomic characterization of the mouse melastatin gene (MIsn1). Genomics 54: 116-123.
Kim, C., 2004 Transient receptor ion channels and animal sensation: lessons from Drosophila functional research. J. Biochem. Mol. Biol. 37: 114-121.
Koike, C., R. Sanuki, K. Miyata, T. Koyasu, T. Miyoshi et al., 2007 The functional analysis of TRPM1 in retinal bipolar cells. Neurosci. Res. 58S: S41.
Komaromy, A. M., S. E. Andrew, H. L. Sapp, Jr., D. E. Brooks and W. W. Dawson, 2003 Flash electroretinography in standing horses using the DTL microfiber electrode. Vet. Ophthalmol. 6: 27-33.
Lapp, R. A., and G. Carr, 1998 Applied appaloosa color genetics. Appaloosa J. 52: 113-115.
Lee, S. T., R. D. Nicholls, R. E. Schnur, L. C. Guida, J. Lu-Kuo et al., 1994 Diverse mutations of the P gene among African-Americans with type II (tyrosinase-positive) oculocutaneous albinism (OCA2). Hum. Mol. Genet. 3: 2047-2051.
Lim et al. (2003) Science.299:1540

Locke, M. M. et al. Penedo, M. C. T., Bricker, S. J., Millon, L. V., & Murray J. D. (2002) Anim Geneti 33, 329-37
MacLeod, J. N., N. Burton-Wurster, D. N. Gu and G. Lust, 1996 Fibronectin mRNA splice variant in articular cartilage lacks bases encoding the V, III-15, and I-10 protein segments. J. Biol. Chem. 271(31): 18954-18960.
Mariat, D., S. Taourit and G. Guerin, 2003 A mutation in the MATP gene causes the cream coat colour in the horse. Genet. Sel. Evol. 35: 119-133.
Marklund, L., M. J. Moller, K. Sandberg and L. Andersson, 1996 Amissensemutation in the gene for melanocyte-stimulating hormone receptor (MC1R) is associated with the chestnut coat color in horses. Mamm. Genome 7: 895-899.
Metallinos, D. L., A. T. Bowling and J. Rine, 1998 A missense mutation in the endothelin-B receptor gene is associated with Lethal White Foal Syndrome: an equine version of Hirschsprung disease. Mamm. Genome 9: 426-431.
Miller, R. W., 1965 Appaloosa coat color inheritance. Ph.D. Thesis, Animal Science Department, Montana State University, Bozeman, Mont.
Murphy, B. A., M. M. Vick, D. R. Sessions, R. F. Cook and B. P. Fitzgerald, 2006 Evidence of an oscillating peripheral clock in an equine fibroblast cell line and adipose tissue but not in peripheral blood. J. Comp. Physiol. A Neuroethol. Sens. Neural Behav. Physiol. 192: 743-751.
Nakanishi, S., Y. Nakajima, M. Masu, Y. Ueda, K. Nakahara et al., 1998 Glutamate receptors: brain function and signal transduction. Brain Res. Rev. 26: 230-235.
Ni-Komatsu, L., and S. J. Orlow, 2006 Heterologous expression of tyrosinase recapitulates the misprocessing and mistrafficking in oculocutaneous albinism type 2: effects of altering intracellular pH and pink-eyed dilution gene expression. Exp. Eye Res. 82: 519-528.
Nilius, B., 2007 TRP channels in disease. Biochim. Biophys. Acta 1772: 805-812.
Nomura, M., H. Iwakabe, Y. Tagawa, T. Miyoshi, Y. Yamashitaet al., 1994 Developmentally regulated postsynaptic localization of a metabotropic glutamate receptor in rat rod biopolar cells. Cell 77: 361-369.
Pfaffl, M. W., 2001 A new mathematical model for relative quantification in real-time RT-PCR. Nucleic Acids Res. 29: e45.
Pfaffl, M. W., G. W. Horgan and L. Dempfle, 2002 Relative Expression software tool (REST) for group-wise comparison and statistical analysis of relative expression results in real-time PCR. Nucleic Acids Res. 30: e36.
Porter, S. D., and C. J. Meyer, 1994 A distal tyrosinase upstream element stimulates gene expression in neural-crest-derived melanocytes of transgenic mice: position-independent and mosaic expression. Development 120: 2103-2111.
Porter, S., L. Larue and B. Mintz, 1991 Mosaicism of tyrosinaselocus transcription and chromatin structure in dark vs. light melanocyte clones of homozygous chinchilla-mottled mice. Dev. Genet. 12: 393-402.
Ray, K., M. Chaki and M. Sengupta, 2007 Tyrosinase and ocular diseases: some novel thoughts on the molecular basis of oculocutaneous albinism type 1. Prog. Retin. Eye Res. 26: 323-358.
Rebhun, W. C., E. R. Loew, R. C. Riis and L. J. Laratta, 1984 Clinical manifestations of night blindness in the Appaloosa horse. Comp. Contin. Edu. Pract. Vet. 6: S103-S106.
Rieder, S., S. Taourit, D. Mariat, B. Langlois and G. Gue′rin, 2001 Mutations in the agouti (ASIP), the extension (MC1R), and the brown (TYRP1) loci and their association to coat color phenotypes in horses (Equus caballus). Mamm. Genome 12: 450-455.
Sandmeyer, L., C. B. Breaux, S. Archer and B. H. Grahn, 2007 Clinical and electroretinographic characteristics of congenital stationary night blindness in the Appaloosa and the association with the leopard complex. Vet. Ophthalmol. 10: 368-375.
Schubert, G., and H. Bornshein, 1952 Beitrag zur A lyse des menschlichen Electroretinogram. Ophthalmolgica 123: 396-413.
Silvers, W. K, 1979 The Coat Colors of Mice. Springer-Verlag, New York.
Sponenberg, D. P., and B. V. Beaver, 1983 Horse Color. Texas A&M Press, College Station, Tex.
Sponenberg, D. P., G. Carr, E. Simak and K. Schwink, 1990 The inheritance of the leopard complex of spotting patterns in horses. J. Hered. 81: 323-331.
Steingrimsson, E., N. G. Copeland and N. A. Jenkins, 2006 Mouse coat color mutations: from fancy mice to functional genomics. Dev. Dyn. 235: 2401-2411.
Stryer, L., 1991 Visual excitation and recovery. J. Biol. Chem. 266: 10711-10714.
Sturm, R. A., R. D. Teasdale and N. F. Box, 2001 Human pigmentation genes: identification, structure and consequences of polymorphic variation. Gene 277: 49-62.
Szabo, V., H. J. Kreienkamp, T. Rosenberg and A. Gal, 2007 Gln200Glu, a putative constitutively active mutant of rod alpha-transducin (GNAT1) in autosomal dominant congenital stationary night blindness. Hum. Mutat. 28: 741-742.
Terry, R. B., S. Archer, S. Brooks, D. Bernoco and E. Bailey, 2004 Assignment of the appaloosa coat colour gene (LP) to equine chromosome 1. Anim. Genet. 35: 134-137.
Toyofuku, K., I. Wada, R. A. Spritz and V. J. Hearing, 2001 The molecular basis of oculocutaneous albinism type 1 (OCA1): sorting failure and degradation of mutant tyrosinases results in a lack of pigmentation. Biochem. J. 355: 259-269.
Witzel, D. A., J. R. Joyce and E. L. Smith, 1977 Electroretinography of congenital night blindness in an Appaloosa filly. J. Eq. Med. Surg. 1: 226-229.
Witzel, D. A., E. L. Smith, R. D. Wilson and G. D. Aguirre, 1978 Congenital stationary night blindness: an animal model. Invest. Ophthalmol. Vis. Sci. 1978(117): 788-793.
Xiao, X., X. Jia, X. Guo, S. Li, Z. Yanget al., 2006 CSNB1 in Chinese families associated with novel mutations in NYX. J. Hum. Genet. 51: 634-640.
Xu, X. Z., F. Moebius, D. L. Gill and C. Montell, 2001 Regulation of melastatin, a TRP-related protein, through interaction with a cytoplasmic isoform. Proc. Natl. Acad. Sci. USA 98: 10692-10697.
Zeitz, C., B. Kloeckener-Gruissem, U. Forster, S. Kohl, I. Magyar et al., 2006 Mutations in CABP4, the gene encoding the Ca2+-binding protein 4, cause autosomal recessive night blindness. Am. J. Hum. Genet. 79: 657-667.
Zhiqi, S., M. H. Soltani, K. M. Bhat, N. Sangha, D. Fang et al., 2004 Human melastatin 1 (TRPM1) is regulated by MITF and produces multiple polypeptide isoforms in melanocytes and melanoma. Melanoma Res. 14: 509-516.

1. A method of screening for, diagnosing or detecting congenital stationary night blindness (CSNB) in a subject comprising the steps:
(a) (i) determining a level of a biomarker product in a sample from the subject, wherein the biomarker is TRPM1; and
(ii) comparing the level of the biomarker product in the sample with a control;
wherein detecting differential expression of the biomarker product between the subject and the control is indicative of congenital stationary night blindness in the subject; or
(b) determining the presence of at least one SNP allele associated with CSNB in the TRPM1 gene.

2. The method according to claim 1, wherein the biomarker product is an RNA product or a protein product.

3. (canceled)

4. The method according to claim 1, wherein the sample comprises retina, retina pigment epithelium, or skin.

5. The method according to claim 1, wherein the control is from a subject known not to have congenital stationary night blindness and the level of biomarker product from the sample from the subject is lower as compared to the control, indicating that the subject has congenital stationary night blindness.

6. (canceled)

7. The method of claim 1, wherein the at least one SNP allele associated with CSNB is:
(a) located at position 108249293 of ECA1, wherein the associated allele is an A nucleotide;
(b) located at position 108267503 of ECA1, wherein the associated allele is a C nucleotide;
(c) selected from the SNPs listed in Table 9;
(d) located at position 108370091 of ECA1, wherein the associated allele is a T nucleotide; or
(e) located at position 108370150 of ECA1, wherein the associated allele is a C nucleotide.

8-13. (canceled)

14. A method of detecting or selecting different coat patterns in a horse comprising the steps:
(a) (i) determining a level of a biomarker product in a sample from the horse, wherein the biomarker is TRPM1; and
(ii) comparing the level of the biomarker product in the sample with a control;
wherein detecting differential expression of the biomarker product in the sample compared to the control is indicative of a different coat pattern; or
(b) determining the presence of at least one SNP allele associated with leopard complex spotting (LP) in the TRPM1 gene in a sample from the horse.

15. The method according to claim 14, wherein the biomarker product is an RNA product or a protein product.

16. (canceled)

17. The method according to claim 14, wherein the sample comprises skin.

18. (canceled)

19. The method of claim 14, wherein the at least one SNP allele associated with LP is:
(a) located at position 108249293 of ECA1, wherein the associated allele is an A nucleotide;
(b) located at position 108267503 of ECA1, wherein the associated allele is a C nucleotide;
(c) selected from the SNPs listed in Table 8; or
(d) located at position 108370091 of ECA1, wherein the associated allele is a T nucleotide.

20-24. (canceled)

25. A kit for screening for, diagnosing or detecting congenital stationary night blindness (CSNB) in a subject comprising:
a) a binding agent that binds a biomarker or a biomarker product, wherein the biomarker is TRPM1, and the binding agent is selected from:
(i) a probe that specifically hybridizes to a SNP allele associated with CSNB or a pair of primers for amplifying a sequence comprising the SNP allele associated with CSNB;
(ii) an antibody that specifically binds the protein biomarker product;
(iii) a probe that specifically hybridizes to the nucleic acid biomarker product;
(iv) primers for amplifying the nucleic acid biomarker product; and
b)instructions for use.

26-28. (canceled)

29. The kit according to claim 25, wherein the probe in (iii) comprises the nucleic acid sequence of SEQ ID NO: 7.

30. The kit according to claim 25, wherein the primers in (iv) comprise the nucleic acid sequence of SEQ ID NOS: 5 and 6.

31. (canceled)

32. A kit for detecting or selecting different coat patterns in a horse comprising:
a) a binding agent that binds a biomarker or a biomarker product, wherein the biomarker is TRPM1, and the binding agent is selected from:
(i) a probe that specifically hybridizes to a SNP allele associated with LP or a pair of primers for amplifying a sequence comprising the SNP allele associated with LP;
(ii) an antibody that specifically binds the protein biomarker product;
(iii) a probe that specifically hybridizes to the nucleic acid biomarker product; or
(iv) primers for amplifying the biomarker product; and
b) instructions for use.

33. (canceled)

34. (canceled)

35. The kit according to claim 32, wherein the probe in (iii) comprises the nucleic acid sequence of SEQ ID NO: 7.

36. The kit according to claim 32, wherein the primers in (iv) comprise the nucleic acid sequence of SEQ ID NOS: 5 and 6.
